(19) United States
(12) Patent Application Publication
(10) Pub. No.: US 2015/0376294 A1
(43) Pub. Date: Dec. 31, 2015

(54) ANTI-PAD2 ANTIBODIES AND TREATMENT OF AUTOIMMUNE DISEASES

(71) Applicant: RIGSHOSPITALET, København Ø (DK)

(72) Inventors: Claus Henrik Nielsen, Lyngby (DK); Dres Damgaard, Copenhagen S (DK)

(21) Appl. No.: 14/649,100

(22) PCT Filed: Dec. 2, 2013
(86) PCT No.: PCT/DK2013/050406

§ 371 Date: Jun. 2, 2015

(30) Foreign Application Priority Data
Dec. 3, 2012 (DK) PA 2012 70747

(51) Int. Cl. C07K 16/40

(52) U.S. Cl. CPC C07K 16/40; C07K 2317/565; C07K 2317/24; C07K 2317/21; C07K 2317/76

(57) ABSTRACT

The present invention relates to anti-peptidylarginine deiminase 2 (PAD2) antibodies and anti-PAD2 antibodies for use in the treatment of autoimmune diseases characterized by extracellular citrullination, such as rheumatoid arthritis (RA). The invention further relates to a method for treatment of an autoimmune disease characterized by extracellular citrullination comprising the administration of a suitable amount of an anti-PAD2 antibody to a subject.

All patent and non-patent references cited in the present application, are incorporated by reference in their entirety.

### FIELD OF INVENTION

The present invention relates to anti-peptidylarginine deiminase 2 (PAD2) antibodies, and anti-PAD2 antibodies for use in the treatment of autoimmune diseases characterized by extracellular citrullination, such as rheumatoid arthritis (RA). The invention further relates to a method for treatment of an autoimmune disease characterized by extracellular citrullination comprising the administration of a suitable (or effective) amount of an anti-PAD2 antibody to a subject.

### BACKGROUND OF INVENTION

Citrullination is a process wherein arginine residues in various proteins are deiminated into citrulline. The process is catalysed by enzymes of the peptidylarginine deiminase (PAD) family. After this conversion, the protein loses positive charge, changes conformation and becomes more susceptible to degradation.

Autoimmune diseases arise from an inappropriate immune response of the body against substances and tissues normally present in the body. In a number of autoimmune diseases citrullination has been suggested to play an important role in the pathogenesis. Such diseases include e.g. rheumatoid arthritis, multiple sclerosis and psoriasis.

Rheumatoid Arthritis (RA) affects 0.5-1% of the adult population worldwide. RA is caused by an autoimmune attack on the synovium followed by chronic inflammation in the synovial joints. A role for the PAD isoforms PAD2 and PAD4 has previously been suggested in RA. PAD2 and PAD4 are both present in the inflamed joint. In RA, PAD enzymes are thought to citrullinate extracellular proteins such as fibrinogen, which may contribute to the pathogenesis and/or result in further disease progression by generation of citrullinated proteins that may induce production of anti-citrullinated protein antibodies (ACPAs) and stimulation of T cells reactive with citrullinated peptides. ACPAs are detectable in the serum years before the onset of arthritis symptoms, and ACPA-positive patients have more extensive joint erosion than ACPA-negative patients.

ACPAs have proved a useful diagnostic marker for RA. Thus approximately 88-96% of ACPA-positive individuals will clinically present as RA patients, while approximately 70-80% of RA patients are ACPA-positive. ACPA-positive and −negative RA are often considered two distinct disease entities with similar symptoms. Associated with ACPA-positive RA are human leukocyte A (HLA) antigens containing the “shared epitope”, capable of comprising citrullinated peptides. Thus, proteins citrullinated by PAD trigger antibody responses as well as T-cell responses in ACPA-positive RA.

PAD2 and PAD4 enzymes have previously been detected in synovial tissue from RA patients and their expression levels were found to be correlated with the intensity of inflammation (Foulquier et al. 2007, Arthritis & Rheumatism 56, 11: 3541-53).

Suzuki et al. disclose that the human PAD4 gene locus is a strong susceptibility locus for rheumatoid arthritis, however none of the other PAD genes were found to be associated with susceptibility to rheumatoid arthritis (Suzuki et al. 2003, Nat. Genet. 34, 4: 395-402).

WO 2010/005293 discloses a short peptide inhibitor capable of inhibiting PAD2 and PAD4 activity. The peptide inhibitor comprises 5-20 amino acids.

WO 2011/050357 discloses a small molecule inhibitor of PAD activity of PAD1, PAD3 and PAD4.

WO 2009/127048 discloses a small molecule inhibitor capable of inhibiting PAD2 activity.

EP1717224 discloses a small molecule inhibitor of PAD4 and WO 2012/026309 discloses antibodies directed against PAD4 for the treatment of rheumatoid arthritis.

### SUMMARY OF INVENTION

The present invention relates to antibodies against peptidylarginine deiminase 2 (PAD2) and their use in the treatment of autoimmune diseases characterized by extracellular citrullination, preferably extracellular hyper-citrullination. The autoimmune disease is in one embodiment selected from the group consisting of rheumatoid arthritis, multiple sclerosis, Sjögren's syndrome and psoriasis.

The invention further relates to a method of treatment of autoimmune diseases characterized by extracellular citrullination comprising the administration of a suitable amount of an anti-PAD2 antibody to a subject in need thereof.

The advantage of using an anti-PAD2 antibody for inhibition of PAD2 activity is that the antibody will inhibit specifically extracellular citrullination mediated by PAD2 and not intracellular citrullination, thus preserving the cells' ability to citrullinate important intracellular targets of PAD2. As a consequence, it is expected that a drug comprising an anti-PAD2 antibody will have fewer side-effects than a small molecule inhibitor of PAD2, thus leading to higher patient compliance and safety.

In addition, an antibody directed at PAD2 potentially has the further advantage of being able to inhibit PAD2 activity at several levels both by direct inhibition of enzyme activity and indirectly by stimulating clearance of PAD2.

### DESCRIPTION OF DRAWINGS

FIG. 1. Determination of titer in PAD2-immunized mice. VØ, HØ and 0Ø refers to the three immunized mice. Rabbit PAD2 (rPAD2) was coated at 0.5 μg/mL. Sera were diluted from 1:1000 and incubated for 1.5 hour at RT. Rabbit anti-mouse IgG was added 1:1000 for 45 min at RT followed by OPD substrate development. The levels are given as OD_{490-650 nm}-units.

FIG. 2. mAbs (monoclonal antibodies) reacting with rPAD2 in western blotting. Culture supernatants from mAbs #1-30, an irrelevant mAb (A) and no mAb (B) were tested 1:1 in diluting buffer against 1 μg/mL rabbit PAD2-unreduced. Rabbit anti mouse IgG (diluted 1:1000) was added, 1 h/RT. Development was performed in carbazole staining solution. Molecular weight markers (kDa) are indicated in the middle of the blot with Novex® Sharp Pre stained Protein Standard. A 4-12% Bis-Tris zoom gel was used.

FIG. 3. Culture supernatants from mAb #1-35 tested on human recombinant (hr) PAD2-coated plates. hrPAD2 was diluted in 2-fold steps from 500 ng/mL. Shown is the absorbance at the coating concentration of 32 ng/mL. HRP-conjugated rabbit anti-mouse, diluted 1:1000, was added for 1 hour at RT, and the plates were developed with OPD substrate. The levels are given as OD_{490-650 nm}-units.

FIG. 4. mAbs reacting with hrPAD2 in western blotting. Culture supernatants from mAb #1-35, irrelevant mAbs (A, B, E), and no mAb (C, D) were tested 1:1 in diluting buffer against 0.5 μg/mL hrPAD2-unreduced. Molecular weight markers (kDa) are indicated in the middle of the blot with Novex® Sharp Pre-stained Protein Standard. A 4-12% zoom gel was used.

FIG. 5. Overview of mAbs tested with respect to: Western Blotting and ELISA. Dark grey indicates strongly reacting. Light grey indicates weakly reacting. White indicates non-reacting.

FIG. 6. Culture supernatants from mAb #1-35 tested on hrPAD2/hrPAD4 coated plates—50 ng/mL. HRP Rabbit anti-mouse (p0260) was added 1:1000 for 1 hour at RT and plates were developed with OPD substrate. The levels are given as OD_{490-650 nm}-units.

FIG. 7. Epitope mapping of anti-PAD2. Different splice variants of PAD2 were evaluated by western blotting. Shown is the reactivity of three selected mAbs (#2, #6 and #34) with WT (full length wild type human PAD2), C254 (amino acids 1-254 of human PAD2), 1385-463 (whole length human PAD2 without the catalytic site), N165 (from amino acid 165 to the C-terminus), N343 (from amino acid 343 to the C-terminus). The mAbs were found to bind in the N-terminal region among amino acids 1-165.

FIG. 8. Inhibitory capacity of anti-PAD2 mAbs. The ability of selected anti-PAD2 mAbs to inhibit citrullination of fibrinogen was tested using human recombinant PAD2 (hrPAD2) as catalyst. (A) Test of the inhibitory capacity of mAbs #2, #3 and #33. A mAb against human complement component 4 C4 (anti-C4) was used as negative control. (B) Test of the inhibitory capacity of mAbs #6, #8 and #10. Anti-C4 was used as control. (C) Test of the inhibitory capacity of culture supernatants (cs) from mAb #9, #12, #31 and #34 was tested. mAbs against chicken complement component 3 (chC3) and SCUBE1 (signal peptide, CUB domain, epidermal growth factor-like protein 1) was used as controls.

### DEFINITIONS

Anti-citrullinated protein antibodies (ACPAs): Anti-citrullinated protein antibodies (ACPA), also known as anti-cyclic citrullinated peptide antibodies (anti-CCP), are autoantibodies that are frequently detected in the blood of rheumatoid arthritis patients. These antibodies recognize amino acid sequences containing citrulline in a variety of proteins. During inflammation, arginine residues in proteins such as vimentin can be enzymatically converted into citrulline residues (citrullination), and, if their shapes are significantly altered, the proteins may be seen as antigens by the immune system, thereby generating an immune response. ACPAs have proved to be powerful biomarkers that allow the diagnosis of rheumatoid arthritis (RA) to be made at a very early stage.

Antibody: The term “antibody” or “antibody molecule” describes a functional component of serum and is often referred to either as a collection of molecules (antibodies or immunoglobulin) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e. consisting of identical antibody molecules) or polyclonal (i.e. consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins.

The terms “antibody” or “antibodies” as used herein are also intended to include fully murine, chimeric, humanized, fully human, bispecific and single chain antibodies, nanobodies, as well as binding fragments of antibodies, such as Fab, Fv fragments or single chain Fv (scFv) fragments, as well as multimeric forms such as dimeric IgA molecules or pentavalent IgM. An antibody may be of human or non-human origin, for example a murine or other rodent-derived antibody, or a chimeric, humanized or reshaped antibody based e.g. on a murine antibody.

Each heavy chain of an antibody typically includes a heavy chain variable region (VH) and a heavy chain constant region. The heavy chain constant region typically includes three domains, referred to as CH1, CH2 and CH3. Each antibody light chain typically includes a light chain variable region (VL) and a light chain constant region. The light chain constant region typically includes a single domain, referred to as CL. The VH and VL regions may be further subdivided into regions of hypervariability (“hypervariable regions”, which may be hypervariable in sequence and/or in structurally defined loops). These are also referred to as complementarity determining regions (CDRs), which are interspersed with regions that are more conserved, termed framework regions (FRs). Each VH and VL typically includes three CDRs and four FRs, arranged from the amino terminus to the carboxy terminus in the following order: FR1, CDR1, FR2, CDR2, FR3, CDR3, FR4. The amino acid residues in the variable regions are often numbered using a standardized numbering method known as the Kabat numbering scheme (Kabat et al. (1991) Sequences of Proteins of Immunological Interest, 5th Ed. Public Health Service, National Institutes of Health, Bethesda, M D, USA).

The specificity of an antibody's interaction with a target antigen resides primarily in the amino acid residues located in the six CDRs of the heavy and light chains (three each; CDR1, CDR2 and CDR3 of the heavy chain variable region (VH); and CDR1, CDR2 and CDR3 of the light chain variable region (VL). The amino acid sequences within CDRs are therefore much more variable between individual antibodies than sequences outside of CDRs. Because CDR sequences are responsible for most antibody-antigen interactions, it is possible to express recombinant antibodies that mimic the properties of a specific naturally occurring antibody, or more generally any specific antibody with a given amino acid sequence, by constructing expression vectors that express CDR sequences from the specific antibody grafted into framework sequences from a different antibody. As a result, it is possible to “humanize” a non-human antibody and still substantially maintain the binding specificity and affinity of the original antibody.

Chimeric antibody: A “chimeric antibody” refers in its broadest sense to an antibody that contains one or more regions from one antibody and one or more regions from one or more other antibodies. As used herein, a “chimeric antibody” is generally an antibody that is partially of human origin and partially of non-human origin, i.e. derived in part from a non-human animal, for example a mouse or other rodent, or an avian species such as a chicken.

Recombinant antibody: The term “recombinant antibody” refers to an antibody that is expressed from a cell or cell line transfected with an expression vector (or possibly more than one expression vector, typically two expression vectors) comprising the coding sequence of the antibody, where said coding sequence is not naturally associated with the cell.

Autoimmune disease is used interchangeably with the term “autoimmune disorder” and is characterized by an inappropriate immune response against own cells or tissue (“self”). In healthy circumstances, the immune system attacks only foreign microorganisms or molecules, but in autoimmune diseases the immune system loses the ability to distinguish between self and non-self (loss of tolerance). Like adaptive immune responses against foreign antigens, autoimmune disorders are believed to be initiated by activation of antigen-specific T cells. The T cells may, in turn, activate self-reactive B cells with production of autoantibodies as a consequence. Both genetic and environmental risk factors contribute to breakage of self-tolerance in most autoimmune diseases.

Fab: The fragment antigen-binding (Fab fragment) is a region on an antibody that binds to antigens. It is composed of one constant and one variable domain of each of the heavy and the light chain. These domains shape the paratope—the antigen-binding site—at the amino terminal end of the monomer. The two variable domains bind the epitope on their specific antigens. In an experimental setting, Fc and Fab fragments can be generated in the laboratory. The enzyme papain can be used to cleave an immunoglobulin monomer into two Fab fragments and an Fc fragment. The enzyme pepsin cleaves below hinge region, so a F(ab′)₂ fragment and a pFc′ fragment is formed. The F(ab′)₂ fragment can be split into two Fab′ fragments by mild reduction.

The variable regions of the heavy and light chains can be fused together to form a single-chain variable fragment (scFv), which is only half the size of the Fab fragment, yet retains the original specificity of the parent immunoglobulin.

Nanobody: A nanobody is also known as a single-domain antibody (sdAb) and is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa), and even smaller than Fab fragments (˜50 kDa, one light chain and half a heavy chain) and single-chain variable fragments (˜25 kDa, two variable domains, one from a light and one from a heavy chain).

scFv: A single-chain variable fragment (scFv) is a fusion protein of the variable regions of the heavy (V_{H}) and light chains (V_{L}) of immunoglobulins, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. Unlike monoclonal antibodies, which are often produced in mammalian cell cultures, scFvs are more often produced in bacteria cell cultures such as E. coli.

Bispecific antibodies: A bispecific monoclonal antibody (BsMAb, BsAb) is an artificial protein that is composed of fragments of two different monoclonal antibodies and consequently binds to two different types of antigen. For example in cancer immunotherapy, where BsMAbs are engineered that simultaneously bind to a cytotoxic cell (using a receptor like CD3) and a target like a tumour cell to be destroyed. Bispecific antibodies include trifunctional antibodies, chemically linked F(ab′)₂ and bi-specific T-cell engager (BiTE) herein.

Medical sign: A medical sign is an objective indication of some medical fact or characteristic that may be detected by a physician during a physical examination or by a clinical scientist by means of an in vitro examination of a patient, e.g. blood testing. Medical signs are different from symptoms, the subjective experiences, such as fatigue, that patients might report to their examining physician. For convenience, signs are commonly distinguished from symptoms as follows: Both are something abnormal, relevant to a potential medical condition, but a symptom is experienced and reported by the patient, while a sign is discovered by the physician during examination or by a clinical scientist by means of an in vitro examination of the patient.

Symptom: A symptom is a departure from normal function or feeling which is noticed by a patient, indicating the presence of disease or abnormality. A symptom is subjective, observed by the patient, and cannot be measured directly. For example, the classical symptoms of RA include swollen, warm, painful and stiff joints, particularly early in the morning on waking or following prolonged inactivity.

### DETAILED DESCRIPTION OF THE INVENTION

### Citrullination

Posttranslational modifications, e.g. phosphorylation, glycosylation and citrullination, are normal processes that occur after protein synthesis and are crucial for the enormous diversity of the proteome. Citrullination (also termed deimination) is a post-translational modification whereby the amino acid arginine is modified to the non-standard residue citrulline. This reaction is catalyzed by a group of peptidylarginine deiminase (PAD) enzymes. Citrullination is important for many intracellular processes (reviewed by e.g. György et al. 2006, Int J Biochem Cell Biol 38: 1662-77). Listed are a few examples:
Citrullination of keratin and filaggrin is crucial for the final stage of keratinocyte differentiation.
Citrullination is involved in maturation of hair cuticle cells with importance in the formation of the rigid structures.
Citrullination of myelin basic protein (MBP) is important for ensuring electrical insulation of myelin sheaths.
PAD citrullinates arginine and methylarginine of histone H3 and H4. This disables histone methylation which regulates transcription of DNA.
Regulation of gene expression
apoptosis

Arginine contains a positively charged guanido group in the side chain, before citrullination, which is replaced by a neutral citrulline ureido group, thus reducing the net-charge of a protein. This might prevent the ability to make ionic interactions with negatively charged side chains resulting in different structure of the protein. Loss of intramolecular interactions allowing a protein to unfold thus makes it more susceptible to proteolytic cleavage. This structural change can for example be seen experimentally for fibrinogen which migrates differently in SDS-PAGE after citrullination.

The function of proteins may be altered by citrullination as seen with citrullinated fibrinogen, which markedly impairs the function of thrombin-catalysed fibrin polymerization and also inhibits fibrin formation. The structural changes in proteins can be so dramatic that they are recognized as non-self-protein by the immune system, which is the case in RA. Diseases in which citrullination has been shown or suggested to play a role include not only RA but also diseases such as multiple sclerosis (MS), Sjögren's syndrome as well as psoriasis.

PAD catalyzes the citrullination reaction in a Ca²⁺ dependent manner. There are five members of the human PAD enzyme family (PAD1, PAD2, PAD3, PAD4 and PAD6). They have a molecular mass of around 75 kDa. These PAD enzymes differ with respect to cellular expression patterns and substrate specificities. For example, PAD2 and PAD4 expression has been shown in RA synovium, synovial fluid cells and has further been detected extracellularly in synovial fluid in RA patients. None of the other family members (PAD1, PAD3 and PAD6) have been detected in the synovial joints among patients with RA (Foulquier et al. 2007, Arthritis Rheum 56; 3541-53).

PAD2 and PAD4 isoforms are likewise expressed in the brain and are present in myelin. It has been hypothesized that PAD2 contributes to destabilization of myelin in Multiple Sclerosis (Musse et al. 2008, Disease Models & Mechanisms 1, 229-240).

PAD1 is mainly expressed in epidermis and uterus and is important for the terminal differentiation of keratinocytes, keratins and filaggrin. PAD2 has been widely detected, notably in brain astrocytes, sweat glands, skeletal muscles, epidermis and macrophages. PAD3 is co-expressed and co-localized with its natural substrate, thrichohyalin, which is a major structural protein of inner root sheath cells of hair follicles. PAD4 expression has so far been detected only in leucocytes e.g. monocytes, eosinophils and neutrophils. PAD4 differ from the other PAD isoforms by also being a nuclear protein. PAD6 is expressed in male and female germ cells.

PAD2 is also known as protein-arginine deiminase type-2 and peptidylarginine deiminase II. The human protein sequence ((UniProt no. Q9Y2J8 (PADI2_HUMAN)) comprises 665 amino acids (aa); cf. SEQ ID NO:1. The rabbit PAD2 sequence G1T837 (G1T837_RABIT) also comprises 665 aa's; cf. SEQ ID NO:2. The mouse sequence comprises 673 aa (Q08642 (PADI2_MOUSE)). The protein sequence is highly conserved amongst species, between human and rabbit, and also mouse (mus musculus).

### Autoimmune Diseases

The present invention in one embodiment relates to an anti-PAD2 antibody for use in the treatment of an autoimmune disease. The autoimmune disease is in particular an autoimmune disease characterized by hyper-citrullination; in particular extracellular hyper-citrullination, and/or wherein citrullination plays a role in the pathogenesis of the disease. Such a disease may in one embodiment be selected from the group consisting of rheumatoid arthritis, Sjögren's syndrome, multiple sclerosis and psoriasis.

Targeting extracellular citrullination with an anti-PAD2 antibody avoids the simultaneous inhibition of intracellular processes where citrullination is important, thus reducing or avoiding the risk of adverse effects associated with such general inhibition of citrullination.

In one embodiment the anti-PAD2 antibody of the present invention is used in the treatment of an autoimmune disease. The autoimmune disease is preferably a disease where extracellular citrullination plays a role in the pathogenesis of said disease, such as a disease selected from the group consisting of rheumatoid arthritis, multiple sclerosis and psoriasis. The treatment may be prophylactic, ameliorative and/or curative.

Autoimmune diseases arise from an inappropriate immune response of the body against substances and tissues normally present in the body or against structurally modified derivatives of said substances and tissues. In other words, the immune system mistakes some part of the body as a pathogen and attacks its own cells.

A number of autoimmune diseases are characterized by elevated levels of citrullinated proteins, which are believed to play an important role in the pathogenesis of the autoimmune disease. Such diseases include rheumatoid arthritis, multiple sclerosis, Sjögren's syndrome and psoriasis; in a particular embodiment rheumatoid arthritis and multiple sclerosis, in particular rheumatoid arthritis.

In one embodiment, there is provided an anti-PAD2 antibody according to the present invention for the treatment of an autoimmune disease, such as an autoimmune disease characterized by extracellular citrullination, such as extracellular hyper-citrullination.

In one embodiment, use of an anti-PAD2 antibody according to the present invention for the manufacture of a medicament for the treatment of an autoimmune disease, such as an autoimmune disease characterized by extracellular citrullination, such as extracellular hyper-citrullination, is provided.

### Rheumatoid Arthritis

Rheumatoid Arthritis (RA) is a systemic autoimmune disease affecting 0.5-1% of the adult population worldwide. RA is caused by an autoimmune attack on the synovium followed by chronic inflammation in the synovial joints. Systemic effects are mainly seen as inflammation in lungs, heart and eyes. Like many autoimmune diseases, RA occurs more frequently in women than in men (3:1 ratio) and disease onset is mostly seen at middle age (40-60 years old).

RA normally affects joints symmetrically. Wrists, fingers, feet, ankles and knees are the most commonly affected joints. The first symptoms to appear include warm and tender joints, morning stiffness, and stiffness in the affected joints if not used for an hour or even less. Later on RA patients may lose range of motion in joints and these may become deform, as a result of long-term inflammation and irreversible bone digestion in the synovial joints.

Genetic factors are estimated to be responsible for at least 50% of the risk of RA development, while environmental factors account for the remainder. RA is a polygenic disease and particularly genes of the major histocompatibility complex (MHC) class II provide a strong risk factor in RA as in many other autoimmune diseases. Certain MHC class II types within the HLA-DR region, are thus linked to RA, and 80% of patients with RA carry the so-called shared epitope, variants of a motif (EQKRAA) which is present in the third hypervariable region of the HLA-DR beta chain with structural effect on the binding cleft in the MCH class II molecule. This confers binding of specific citrullinated peptides and thus affects antigen presentation to T-cell receptors. The motif is present in the DRB1* variants *0101, *0102, *0401, *0404, *0405, *0408, *1001 and *1402, which have been associated with RA. The different alleles are associated with mild or a more erosive disease; thus they are likely to present antigens differently, leading to different phenotypes of RA—all with presence of anti-citrullinated protein antibodies (ACPAs). DR1 is associated with a relatively mild disease, whereas DR4 is associated with more severe RA.

A number of single nucleotide polymorphisms (SNPs) have also been associated with RA. Some of these, including TRAF1-C5-, PTPN22- and PAD4 polymorphisms, are also associated with the presence of ACPAs. These factors constitute a smaller risk compared to the MHC-associated risk factors, however. The “non-MHC” risk factors may indicate some of the mechanisms associated with ACPA-negative RA.

Cigarette smoking is the best known environmental risk factor for RA. Several studies link smoking and the shared epitope together as a major combined risk factor. A recent study has shown that smoking increases the expression of PAD2 in bronchoalveolar lavage (BAL) cells along with an elevated level of citrullinated proteins (Makrygiannakis et al., Ann Rheum Dis 67; 1488-92).

The pathogenesis of RA is not clear, but over the last two decades, more insight into pathogenic pathways of RA has accumulated. Autoantigens, e.g. citrullinated peptides, are presented to T cells by antigen presenting cells (APCs) such as dendritic cells, macrophages or activated B cells. This autoantigen-presentation triggers the stimulation and expansion of antigen-specific T cells present in the joints and lymph nodes. Co-stimulatory signals, e.g. CD80 and CD86 presented on APCs, are needed for full activation of the T cells. These bind to surface expressed CD28 on T cells.

T cells localized to the synovial membrane secrete Interleukin-2 (IL-2) and interferon-γ (INF-γ). These cytokines induce activation of macrophages, B cells, fibroblasts and osteoclasts. B cells differentiate into (auto)antibody-secreting plasma cells. The immune complexes containing autoantibodies induce the secretion of proinflammatory cytokines, such as tumor necrosis factor α (TNF-α) via complement- and Fc-receptor mediated activation on human monocytes. Activated B cells also serve as APCs, leading to additional T-cell activation, which enhances the autoimmune response. T- and B cell activation result in increased production of cytokines and chemokines, leading to a feedback loop for additional activation of T cells, macrophages and B cells. Th1 cells activate monocytes and macrophages by cell-cell contact and/or by activation of different cytokines, such as INF-γ, TNF-α and IL-17. The macrophages and fibroblasts then overproduce proinflammatory cytokines, mainly TNF-α, IL-1 and IL-6, which activate osteoclasts (leading to bone destruction) and synovial fibroblasts (leading to production of matrix metalloproteinases and consequent cartilage destruction). A broad range of cytokines are present in the synovium, secreted by various cell populations. The cytokines that have been established to be most directly implicated in RA pathogenesis are TNF-α, IL-6, IL-1, IL-15, IL-18 and IL-17.

There is no known cure for rheumatoid arthritis, but many different types of treatment can alleviate symptoms and/or modify the disease process. Pharmacological treatment of RA can be divided into disease-modifying antirheumatic drugs (DMARDs), biologics, anti-inflammatory agents and analgesics. Treatment also includes rest and physical activity.

In order to be effective, DMARDs must be administered before the deformities appear or the erosive disease occurs. Usually, Rheumatologists do not wait for the fulfilment of the criteria for classification of RA as published by the American College of Rheumatology (ACR) and start treatment with this type of drugs if the pain and synovitis persist and the function is compromised.

DMARDs include but are not limited to: azathioprine, ciclosporin (cyclosporine A), D-penicillamine, gold salts, hydroxychloroquine, leflunomide, methotrexate (MTX), minocycline, sulfasalazine (SSZ) and cyclophosphamide.

Biological agents (biologics) for RA treatment include but are not limited to: tumor necrosis factor alpha (TNFα) blockers (etanercept (Enbrel), infliximab (Remicade), adalimumab (Humira), certolizumab pegol (Cimzia), golimumab (Simponi)), Interleukin 1 (IL-1) blockers (anakinra (Kineret)), monoclonal antibodies against B cells (rituximab (Rituxan)), T-cell costimulation blockers (abatacept (Orencia)), blockers of IL-6 signaling (tocilizumab (an anti-IL-6 receptor antibody) (RoActemra, Actemra)).

Anti-inflammatory agents include but are not limited to glucocorticoids and non-steroidal anti-inflammatory drugs (NSAIDs, most also act as analgesics), and classical analgesics include but are not limited to paracetamol, opiates, diproqualone and lidocaine topical.

NSAIDs used in the treatment of RA include but are not limited to ibuprofen, naproxen, meloxicam, etodolac, nabumetone, sulindac, tolementin, choline magnesium salicylate, diclofenac, diflusinal, indomethicin, ketoprofen, oxaprozin, and piroxicam.

As mentioned, a role for PAD2 and PAD4 has previously been suggested in RA. PAD2 and PAD4 are both present at sites of inflammation, e.g. an inflamed joint. However, PAD2 and PAD4 differ with respect to citrullination efficiency of different substrates. Most proteins can be citrullinated by more than one PAD isoform, but they seem to have substrate-preferences and differ with respect to conditions required for efficient catalysis. For instance, fibrinogen can be citrullinated by either one of PAD2 or PAD4. However, PAD2 is able to citrullinate additional arginine residues compared to PAD4, and the two isoforms differ in enzymatic activity with regard to optimal pH and calcium concentrations. It seems that the amino acid context is more important for PAD4 activity than for PAD2. Furthermore, PAD2 is able to citrullinate fibrinogen much more efficiently than PAD4. Both of PAD2 and PAD4 are able to citrullinate histone H3, however PAD4 is more efficient than PAD2, which is likely a result of PAD4 being present in the nucleus.

Inflammation in the joints leads to infiltration of inflammatory cells. Inflammation is strictly controlled and does not normally lead to production of autoantibodies or auto-reactive T cells. Inflammation is linked with apoptosis of cells, and the apoptotic cells are normally cleared by phagocytes. Massive apoptosis due to toxins, infections or defects in the clearing system, e.g. genetic defects, may result in necrosis of some cells, however. In a joint with inflammation and apoptosis, the intracellular concentration of calcium may be elevated enough to activate PAD enzymes in PAD-containing cells, e.g. monocytes, granulocytes and macrophages. Cytosolic proteins like vimentin, which undergo citrullination, will normally not be exposed to the immune system. When cells become necrotic, i.e. when inflammation is uncontrolled, intracellular components like citrullinated proteins can be found extracellularly in the synovial joints. PAD has likewise been detected extracellularly in synovial joints. PAD enzymes are thought to citrullinate extracellular proteins such as fibrinogen, which may contribute to the pathogenesis of RA and/or result in further disease progression by generation of extracellular citrullinated proteins that may lead to the production of anti-citrullinated protein antibodies (ACPAs) and further inflammation.

ACPAs are detectable in the serum years before the onset of arthritis symptoms, and a significant positive correlation exists between the serum titer and clinical, biologic, and radiologic data related to RA activity and severity. ACPA-positive patients have a more erosive disease than those patients that are ACPA-negative. Moreover, ACPA-positive and ACPA-negative RA patients differ with respect to environmental risk factors. A strong association between RA and HLA types containing the shared epitope exists in ACPA-positive RA, particularly in smokers. Taken together, these findings support the notion that ACPA-positive and ACPA-negative RA are actually two different disease entities.

In a particular embodiment an anti-PAD2 antibody of the present invention is used in the treatment of rheumatoid arthritis, in one embodiment ACPA-positive RA. The treatment may be prophylactic, ameliorative and/or curative.

In another embodiment, an anti-PAD2 antibody is used in the treatment of ACPA-negative rheumatoid arthritis.

In one embodiment the use of an anti-PAD2 antibody of the present invention for the manufacture of a medicament for the treatment of rheumatoid arthritis is provided.

In one embodiment the treatment is prophylactic and can be initiated before symptoms of RA appear. For example, the treatment may be initiated upon detection of ACPAs in a blood sample obtained from a patient.

In one embodiment the anti-PAD2 antibody of the present invention is co-administered with another RA drug, such as a DMARD, a biological agent, an anti-inflammatory agent and/or analgesics. The co-administration may be simultaneous, sequential and/or separate.

### Multiple Sclerosis

Multiple sclerosis (MS), also known as “disseminated sclerosis” or “encephalomyelitis disseminata”, is an autoimmune disease in which the fatty myelin sheaths around the axons of the brain and spinal cord are damaged, leading to demyelination and scarring as well as a broad spectrum of signs and symptoms. Disease onset usually occurs in young adults, and it is more common in women. It has a prevalence that ranges between 2 and 150 per 100,000.

MS affects the ability of nerve cells in the brain and spinal cord to communicate with each other effectively. In MS, the body's own immune system attacks and damages the myelin. When myelin is lost, the axons can no longer effectively conduct signals. Although much is known about the mechanisms involved in the disease process, the cause remains unknown. Theories include genetics or infections. Different environmental risk factors have also been found.

There is no known cure for multiple sclerosis. Treatments attempt to return function after an attack, prevent new attacks, and prevent disability. MS medications can have severe adverse effects or be poorly tolerated. The prognosis is difficult to predict; it depends on the subtype of the disease, the individual's disease characteristics, the initial symptoms and the degree of disability the person experiences as time advances.

PAD2 and PAD4 are expressed in the brain, and de-regulated citrullination has been suggested to play a role in the pathogenesis of multiple sclerosis; hence the present authors suggest inhibition of PAD2 according to the present invention, i.e. via an anti-PAD2 antibody.

In one embodiment the anti-PAD2 antibody of the present invention is used in the treatment of multiple sclerosis.

In one embodiment the use of an anti-PAD2 antibody of the present invention for the manufacture of a medicament for the treatment of multiple sclerosis is provided.

In one embodiment the anti-PAD2 antibody of the present invention is co-administered with another drug for the treatment of multiple sclerosis. The co-administration may be simultaneous, sequential and/or separate.

As previously mentioned, a major advantage of using an antibody to inhibit PAD2 compared to e.g. a small molecule inhibitor is that intracellular citrullination essential for production of functional myelin is not affected.

However, for an antibody drug to be able to reach the sclerotic lesions in the brain it is essential that the drug is able to cross the blood brain barrier. It has been reported that the blood-brain barrier is weakened in MS, wherefore it is likely that even an unmodified antibody drug will be able to cross the blood brain barrier in MS patients.

To treat MS in its early stages, it may however be advantageous to genetically engineer the anti-PAD2 antibody of the present invention to ensure that the antibody is capable of crossing the blood-brain barrier, e.g. by making a bispecific antibody with one “arm” directed against a receptor which transports the antibody across the blood-brain barrier and the other “arm” directed against the target itself, as previously described (Pardridge et al. 2012, Methods Enzymol 503:269-92).

Another strategy which can be undertaken to make antibody drugs capable of crossing the blood-brain barrier is to link the antibody to a peptide capable of crossing the blood-brain barrier, such as viral Tat.

In one embodiment the present invention relates to a modified anti-PAD2 antibody capable of crossing the blood-brain barrier, such as a bispecific anti-PAD2 antibody or an anti-PAD2 antibody linked to Tat. Methods for developing bispecific antibodies capable of crossing the blood-brain barrier are known in the art as are methods for linking antibodies to other proteins/peptides.

### Psoriasis

Psoriasis is an autoimmune disease that affects the skin. It occurs when the immune system mistakes the skin cells for a pathogen, and sends out faulty signals that speed up the growth cycle of skin cells. There are five types of psoriasis: plaque, guttate, inverse, pustular, and erythrodermic. The most common form, plaque psoriasis, is commonly seen as red and white hues of scaly patches appearing on the top first layer of the epidermis (skin). The cause and pathogenesis of psoriasis is not fully understood.

Hyper-citrullination has been suggested to play a role in the pathogenesis of psoriasis; hence the present inventors suggest inhibition of PAD2 according to the present invention, i.e. via an anti-PAD2 antibody.

In one embodiment the anti-PAD2 antibody of the present invention is used in the treatment of psoriasis.

In one embodiment the use of an anti-PAD2 antibody of the present invention for the manufacture of a medicament for the treatment of psoriasis is provided. In one embodiment the anti-PAD2 antibody of the present invention is co-administered with another drug for the treatment of psoriasis. The co-administration may be simultaneous, sequential and/or separate.

Sjögren's Syndrome

Sjögren's syndrome is a systemic autoimmune disease in which immune cells attack and destroy the exocrine glands that produce tears and saliva. It is estimated to affect as many as 4 million people in the United States alone, making it the second most common rheumatic disease.

Sjögren's syndrome can exist as a disorder in its own right (primary Sjögren's syndrome) or may develop years after the onset of an associated rheumatic disorder, such as rheumatoid arthritis, systemic lupus erythematosus, scleroderma, primary biliary cirrhosis etc. (secondary Sjögren's syndrome). Sjögren's syndrome frequently occurs secondary to rheumatoid arthritis.

Blood tests can be done to determine if a patient has high levels of antibodies that are indicative of the condition, such as anti-nuclear antibody (ANA) and rheumatoid factor (because SS frequently occurs secondary to rheumatoid arthritis), which are associated with autoimmune diseases. Around 10% of the patients produce ACPAs.

The pathogenesis of Sjögren's Syndrome is not well understood. At present, there is no cure for Sjögren's syndrome, nor does a specific treatment exist to permanently restore gland secretion.

Increased levels of PAD2 and citrullinated proteins have been detected in salivary glands from patients with Sjögrens Syndrome, as compared to healthy controls. Citrullination may be a determining factor in the autoimmune response in Sjögrens syndrome, at least in a proportion of the patients with ACPAs. Hence, inhibition of PAD2 by means of an anti-PAD2 antibody is potentially a therapeutic approach applicable for Sjögrens syndrome.

In one embodiment the anti-PAD2 antibody of the present invention is used in the treatment of Sjögren's syndrome.

Comorbidity is the presence of one or more additional disorders or diseases co-occurring with a primary disease or disorder; or the effect of such additional disorders or diseases. Sjögren's syndrome frequently occurs secondary to rheumatoid arthritis. Thus, in one embodiment the anti-PAD2 antibody of the present invention is used in the treatment of rheumatoid arthritis and Sjögren's syndrome.

In one embodiment the use of an anti-PAD2 antibody of the present invention for the manufacture of a medicament for the treatment of Sjögren's syndrome is provided.

In one embodiment the anti-PAD2 antibody of the present invention is co-administered with another drug for the treatment of Sjögren's syndrome. The co-administration may be simultaneous, sequential and/or separate.

### Antibody Directed Against PAD2

The present invention in one embodiment relates to an antibody against peptidylarginine deiminase 2 (PAD2) for use in the treatment of an autoimmune disease characterized by extracellular citrullination, in a preferred embodiment extracellular hyper-citrullination. The autoimmune disease is in one embodiment selected from the group consisting of rheumatoid arthritis, multiple sclerosis, Sjögren's syndrome and psoriasis. In a particular embodiment, the autoimmune disease is rheumatoid arthritis.

The invention further relates to a method of treatment of autoimmune diseases characterized by extracellular citrullination comprising the administration of a suitable or effective amount of an anti-PAD2 antibody to a subject in need thereof.

The advantage of using an anti-PAD2 antibody for inhibition of PAD2 activity is that the antibody will inhibit specifically extracellular citrullination mediated by PAD2 and not intracellular citrullination, thus preserving the cells' ability to citrullinate important intracellular targets of PAD2. As a consequence, it is expected that a drug comprising an anti-PAD2 antibody will have fewer side-effects than other inhibitors of PAD2 e.g. a small molecule inhibitor of PAD2, thus leading to higher patient compliance and safety. In addition, an antibody directed at PAD2 has the further advantage of potentially being able to inhibit PAD2 activity at several levels both by direct inhibition of enzyme activity and by stimulating clearance of PAD2.

The anti-PAD2 antibody of the present invention preferably binds specifically to PAD2 and not to the other PAD enzyme isoforms, hence in a preferred embodiment the anti-PAD2 antibody of the present invention does not bind to any one of PAD1, PAD3, PAD4 and PAD6.

Methods of determining “intertarget” specificity of antibodies are known in the art and include e.g. ELISA, western blotting and immunohisto- and/or cytochemistry techniques.

### Epitope Mapping

It may also be of interest to determine the specific epitope of PAD2 which is bound by the anti-PAD2 antibody. Such “intra-target” specificity of the anti-PAD2 antibody may be determined by e.g. epitope mapping. Epitope mapping may be performed by a number of methodologies, which do not necessarily exclude each other. One way to map the epitope-specificity of an antibody molecule is to assess the binding to peptides of varying lengths derived from the primary structure of the target antigen. Such peptides may be both linear and conformational and may be used in a number of assay formats, including ELISA, FLISA and surface plasmon resonance (SPR, Biacore, FACS). Furthermore, the peptides may be rationally selected using available sequence and structure data to represent e.g. extracellular regions or conserved regions of the target antigen, or may be designed as a panel of overlapping peptides representing a selected part or all of the antigen. Specific reactivity of an antibody clone with one or more such peptides will generally be an indication of the epitope specificity. However, peptides are in many cases poor mimics of the epitopes recognized by antibodies raised against proteinaceous antigens, both due to a lack of natural or specific conformation and due to the generally larger buried surface area of interaction between an antibody and a protein antigen as compared to an antibody and a peptide. A second method for epitope mapping, which allows for the definition of specificities directly on the protein antigen, is by selective epitope masking using existing, well defined antibodies. Reduced binding of a second, probing antibody to the antigen following blocking is generally indicative of shared or overlapping epitopes. Epitope mapping by selective masking may be performed by a number of immunoassays, including, but not restricted to, ELISA and Biacore, which are well known in the art. Yet another potential method for the determination of the epitope specificity of anti-PAD2 antibodies is the selection of escape mutants in the presence of antibody. This can e.g. be performed using an alanine-scan. Sequencing of the gene(s) of interest from such escape mutants will generally reveal which amino acids in the antigen(s) that are important for the recognition by the antibody and thus constitute (part of) the epitope.

The present inventors have shown that three mAbs (#2, #6 and #34) retain reactivity with WT (full length wild type human PAD2), C254 (amino acids 1-254 of human PAD2) and 1385-463 (whole length human PAD2 without the catalytic site). However, reactivity was absent with N165 (from amino acid 165 to the C-terminus) and N343 (from amino acid 343 to the C-terminus), thus leading to the conclusion that the mAbs bind in the N-terminal region of PAD2. A typical epitope is approx. 8-10 amino acids in length, and thus it may be expected that the epitope lies at least within the first 1-175 amino acids of PAD2.

The present invention in one embodiment provides an anti-PAD2 antibody, or a functional fragment thereof, that recognizes and specifically binds to a PAD2 epitope located within amino acids 1 to 175 of human PAD2 (SEQ ID NO:1), such as within amino acids 1 to 165 of PAD2 (SEQ ID NO:1).

The present invention in one embodiment provides an anti-PAD2 antibody that recognizes and specifically binds to a PAD2 epitope located within amino acids 1 to 175 of human PAD2 (SEQ ID NO:1), such as within amino acids 1 to 165 of PAD2 (SEQ ID NO:1), for use in the treatment of an autoimmune disease, such as an autoimmune disease characterized by extracellular citrullination.

### Inhibition of PAD2

The anti-PAD2 antibody of the present invention may directly inhibit the catalytic activity of PAD2. In one embodiment anti-PAD2 antibody directly inhibit the catalytic activity of PAD2, e.g. by binding an epitope of PAD2 situated in or near the active catalytic site of PAD2 or by binding in any other area that affect catalytic activity of PAD2, thereby preventing or reducing interaction of PAD2 with extracellular target proteins. Hence in one embodiment, the anti-PAD2 antibody of the present invention inhibits citrullination by direct inhibition of PAD2 catalytic activity.

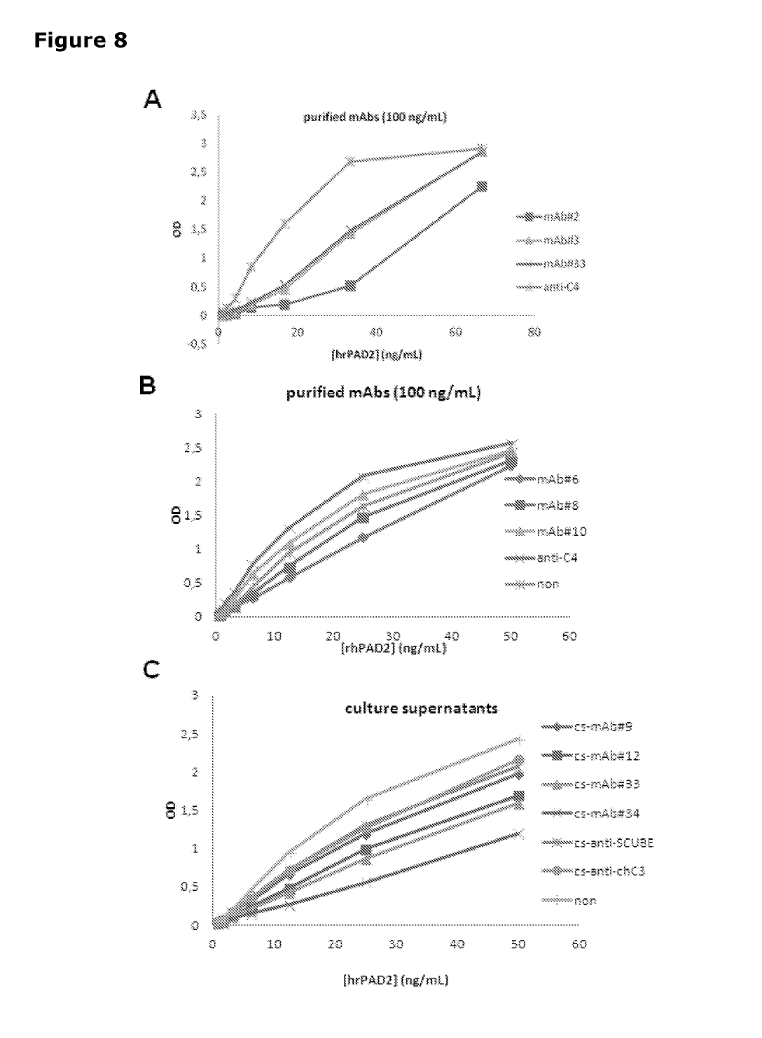

In one embodiment the anti-PAD2 antibody of the present invention inhibits PAD2-catalyzed citrullination of a substrate, such as fibrinogen. In one embodiment the anti-PAD2 antibody inhibits citrullination of fibrinogen with human recombinant PAD2 (hrPAD2) as catalyst (cf. FIG. 8/Example 4).

Catalytic activity of PAD2 and hence the inhibitory effect of anti-PAD2 antibodies on PAD2 catalytic activity can be tested by methods known in the art e.g. by using the commercially available antibody-based PAD enzyme activity assay from ModiQuest Research.

In another embodiment, the anti-PAD2 antibody of the present invention inhibits calcium binding and dimerization, which are required for optimal PAD2 activity.

Inhibition of the catalytic activity of PAD2 is not necessarily required for efficient inhibition of citrullination mediated by PAD2. For example antibody binding to PAD2 may lead to activation of the complement system resulting in efficient clearance of PAD2 and in this way lead to a decrease in the extracellular citrullination levels.

Hence in one embodiment, the anti-PAD2 antibody of the present invention leads to an increased clearance of PAD2. Clearance of PAD2 from blood and extracellular fluid can occur via Fc receptor-mediated endocytosis by phagocytic cells, or (as a consequence formation of complement-activating immune complexes with PAD2) via binding to complement receptors CD35, CD11b/CD18, CD11c/CD18, on phagocytic cells.

In one embodiment the anti-PAD2 antibody of the present invention inhibits the enzyme activity or catalytic activity of PAD2; and/or increases clearance of PAD2.

Also disclosed is a method of inhibiting PAD2-catalyzed citrullination of a substrate, such as fibrinogen, with human recombinant PAD2 (hrPAD2) as catalyst, comprising administration of an anti-PAD2 antibody according to the present invention.

It is well-known in the art that antibodies exist as different isotypes: IgA, IgD, IgE, IgG and IgM. The human IgG and IgA isotypes can be further divided into subclasses IgG1, IgG2, IgG3, IgG4, IgAI and IgA2, whereas the murine IgG isotype can be subdivided into subclasses IgG1, IgG2a, IgG2b, IgG3.

In one embodiment, the antibody isotype is selected from the group consisting of IgA, IgD, IgG and IgM, preferably IgG or IgA, even more preferred IgG. In one embodiment, the antibody isotype is not IgE.

The four IgG subclasses (IgG1, 2, 3, and 4) in humans are named in order of their abundance in serum (IgG1 being the most abundant).

TABLE 1
IgG subclasses

| | Crosses | Complement | Binds to Fc receptor on |
|---|---|---|---|
| Name | placenta easily | activator | phagocytic cells |
| IgG1 | yes | second-highest | high affinity |
| IgG2 | no | third-highest | Extremely low affinity |
| IgG3 | Yes | highest | high affinity |
| IgG4 | yes | no | intermediate affinity |

In one embodiment the anti-PAD2 antibody of the present invention is IgG1.

In one embodiment the anti-PAD2 antibody of the present invention is IgG3.

A potential advantage of using IgG1 and IgG3 isotypes is that they efficiently activate the complement system and bind to Fc receptors on phagocytic cells with high affinity. As a result, said isotype subclasses lead to efficient clearance of target molecule, i.e. PAD2. The Fc-part of an IgG antibody allows salvage of the antibody through the neonatal Fc receptor in the pathway of endocytosis in endothelial cells. Fc receptors in the acidic endosomes bind to IgG internalized through pinocytosis, recycling it to the cell surface, releasing it at the basic pH of blood, thereby preventing it from undergoing lysosomal degradation. This mechanism prolongs the half-life of IgG in the blood compared to other isotypes, and conjugation of some drugs to the Fc domain of IgG significantly increases their half-life.

The anti-PAD2 antibody of the invention is in one embodiment a theraupeutic antibody drug for use in the treatment of an autoimmune disease as defined herein, such as RA. The therapeutic antibody drug may e.g. be a monoclonal therapeutic antibody or a polyclonal therapeutic antibody.

In one embodiment the anti-PAD2 antibody of the present invention is a recombinant antibody.

In other embodiments, the anti-PAD2 antibody drug of the present invention is selected from the group consisting of: a fully non-human (e.g. murine) antibody, a chimeric (e.g. human-mouse) antibody, a humanized antibody, a drug comprising one or more Fab fragments, a nanobody, a single-chain Fv drug (scFv), and a bispecific antibody.

In one embodiment the anti-PAD2 antibody of the present invention is a fully non-human antibody, such as a murine monoclonal antibody. Methods for producing antibodies in e.g. mice, rabbits and other animals are well-known in the art.

In one embodiment the anti-PAD2 antibody is a chimeric antibody. Chimeric antibodies are generally preferred over non-human antibodies in order to reduce the risk of a human anti-antibody response, e.g. a human anti-mouse antibody response in the case of a murine antibody. An example of a typical chimeric antibody is one in which the variable region sequences are murine sequences derived from immunization of a mouse, while the constant region sequences are human. In the case of a chimeric antibody, the non-human parts, i.e. typically the framework regions of the variable region sequences, may be subjected to further alteration in order to humanize the antibody. Methods for producing chimeric antibodies based on the sequence of a non-human antibody are well-known in the art and are described by e.g. Chintalacharuvu et al. 1995. Chimeric Antibodies: Production and Applications. Methods: 8(2); 73-82.

In one embodiment the anti-PAD2 antibody of the present invention is a humanized antibody. Humanized antibodies are approximately 90-95% human and 5-10% non-human, e.g. mouse. The term “humanize” refers to the fact that where an antibody is wholly or partially of non-human origin, for example a murine antibody obtained from immunization of mice with an antigen of interest or a chimeric antibody based on such a murine antibody, it is possible to replace certain amino acids, in particular in the framework regions and constant domains of the heavy and light chains, in order to avoid or minimize an immune response in humans. It is known that all antibodies have the potential for eliciting a human anti-antibody response, which correlates to some extent with the degree of “humanness” of the antibody in question. Although it is not possible to precisely predict the immunogenicity and thereby the human anti-antibody response of a particular antibody, non-human antibodies tend to be more immunogenic than human antibodies. Chimeric antibodies, where the foreign (usually rodent) constant regions have been replaced with sequences of human origin, have been shown to be generally less immunogenic than antibodies of fully foreign origin, and the trend in therapeutic antibodies is towards humanized or fully human antibodies. For chimeric antibodies or other antibodies of non-human origin, it is therefore preferred that they be humanized to reduce the risk of a human anti-antibody response.

For chimeric antibodies, humanization typically involves modification of the framework regions of the variable region sequences. Amino acid residues that are part of a complementarity determining region (CDR) will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, preferably by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues.

Numerous methods for humanization of an antibody sequence are known in the art; see e.g. the review by Almagro & Fransson (2008) Front Biosci. 13: 1619-1633. One commonly used method is CDR grafting, which for e.g. a murine-derived chimeric antibody involves identification of human germline gene counterparts to the murine variable region genes and grafting of the murine CDR sequences into this framework. CDR grafting may be based on the Kabat CDR definitions. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues.

In certain cases, it may also be desirable to alter one or more CDR amino acid residues in order to improve binding affinity to the target epitope. This is known as “affinity maturation” and may optionally be performed in connection with humanization, for example in situations where humanization of an antibody leads to reduced binding specificity or affinity and it is not possible to sufficiently improve the binding specificity or affinity by back mutations alone. Various affinity maturation methods are known in the art, for example the in vitro scanning saturation mutagenesis method described by Burks et al. (1997) PNAS USA, vol. 94, pp. 412-417 and the stepwise in vitro affinity maturation method of Wu et al. (1998) PNAS USA, vol. 95, pp. 6037-6042.

In one embodiment, the anti-PAD2 antibody is a fully human antibody.

In one embodiment the anti-PAD2 antibody of the present invention is a bispecific antibody, e.g. an antibody capable of binding to two different epitopes, wherein said epitopes are present on the same or on different antigens. A bispecific antibody according to the present invention may e.g. comprise antigen-binding regions capable of binding to two different epitopes of PAD2 analogous to e.g. the bispecific antibodies described in e.g. WO 2012/143523.

A bispecific antibody according to the present invention may also comprise one antigen-binding region capable of binding PAD2 and another antigen-binding region capable of binding to a different antigen e.g. to target the bispecific antibody to a preferred site of action. For example the other antigen may e.g. be a synovial membrane protein in case of RA or a blood-brain barrier protein in case of MS, as described previously.

Nanobodies (single-domain antibodies (sdAbs)) are antibody fragments consisting of a single monomeric variable antibody domain. Like a whole antibody, it is able to bind selectively to a specific antigen. With a molecular weight of only 12-15 kDa, single-domain antibodies are much smaller than common antibodies (150-160 kDa), and even smaller than Fab fragments (˜50 kDa, one light chain and half a heavy chain) and single-chain variable fragments (˜25 kDa, two variable domains, one from a light and one from a heavy chain). These peptides have similar affinity to antigens as whole antibodies, but are more heat-resistant and stable towards detergents and high concentrations of urea. The comparatively low molecular mass leads to a better permeability in tissues, and to a short plasma half-life since they are eliminated renally. Unlike whole antibodies, they do not show complement system triggered cytotoxicity because they lack an Fc region. Camelid and fish derived sdAbs are able to bind to hidden antigens that are not accessible to whole antibodies, for example to the active sites of enzymes. This property has been shown to result from their extended CDR3 loop, which is able to penetrate such sites.

In an alternative embodiment the anti-PAD2 antibody of the present invention is a fragment of an antibody, such as a Fab-fragment, a single-chain variable fragment (scFv) or a nanobody.

The present inventors have generated 35 hybridoma clones producing monoclonal antibodies (mAbs) against rabbit PAD2 (rPAD2). The antibodies shall be referred to as mAbs 1-35 herein.

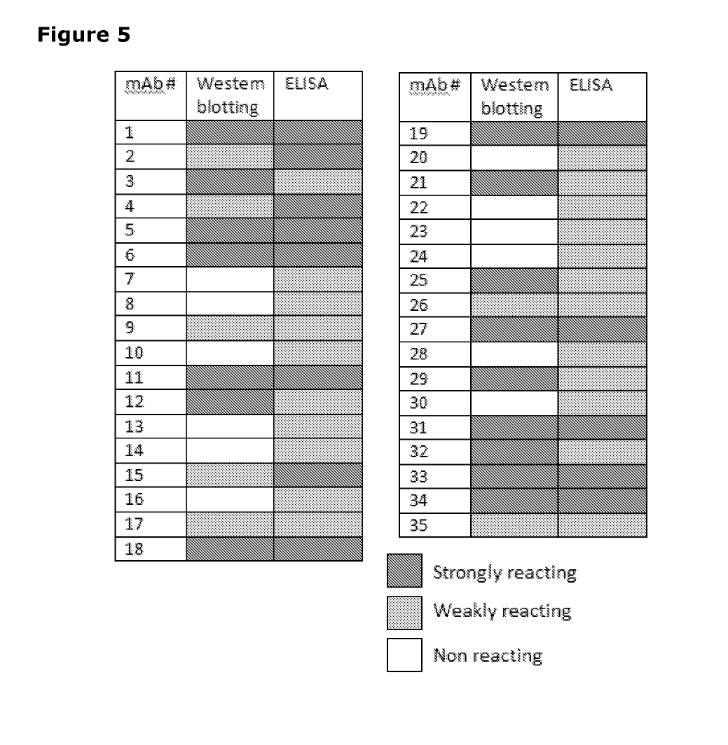

The anti-PAD2 antibodies mAbs 1-35 were tested for their reactivity with rPAD2 and cross-reactivity with human PAD2 (hPAD2). FIG. 5 shows an overview of the ability of the 35 mAbs to recognize hPAD2 by ELISA and western blotting experiments.

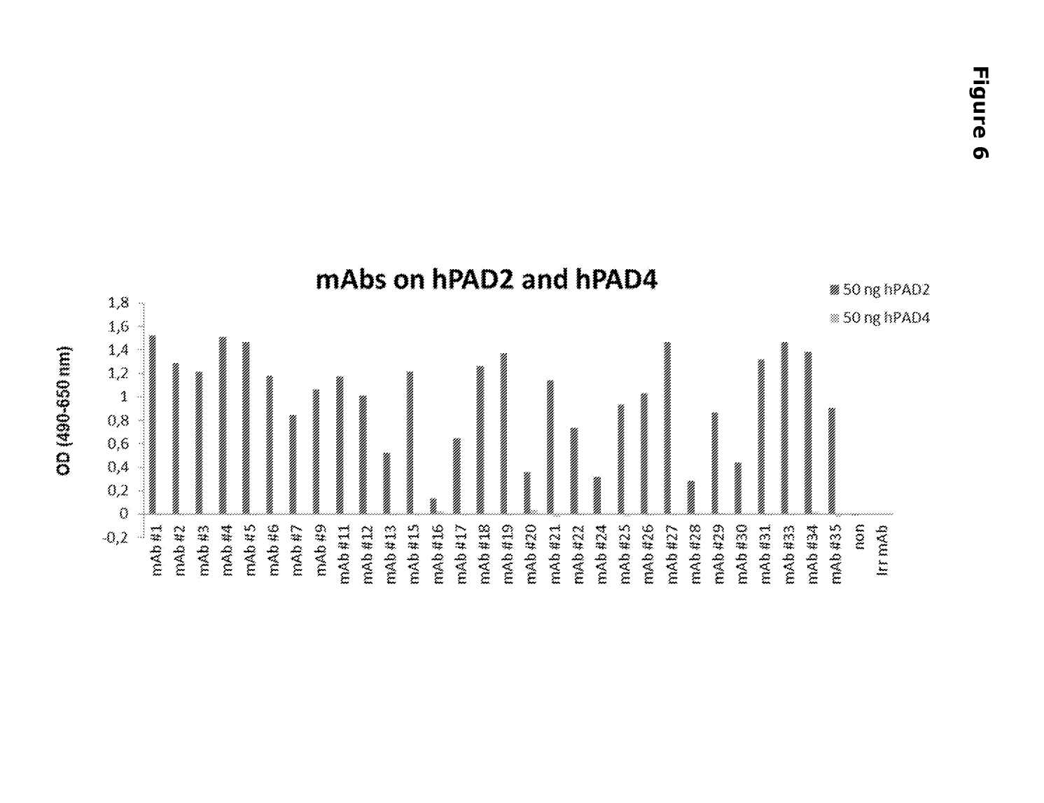

The anti-PAD2 antibodies mAbs 1-35 are specific for the PAD2 isoform as the mAbs did not recognise human PAD4 (hPAD4) (FIG. 6).

In one embodiment the present invention relates to an anti-PAD2 antibody selected from the group consisting of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and mAb 35.

Knowing the sequence of the monoclonal antibodies is the first step towards antibody engineering and function optimization. Sequencing of monoclonal antibodies is state of the art and automated antibody-sequencing services are performed by several companies. Sequencing of monoclonal antibodies may e.g. be performed by a method comprising the steps of: mRNA isolation, reverse transcription, PCR amplification of heavy and light chains, cloning into a standard sequencing vector; sequencing. Full-length antibody sequencing, CDR sequencing and characterization of antibodies may also be performed using a combination of N-terminal and internal Edman protein sequencing, capillary LC with microfraction collector, MS and MSMS mass spectrometry and de novo peptide sequencing. LC and HC fragments of purified antibody are separately digested by several cleavage methods into peptides to get overlapping amino acid peptide sequences of the protein. The peptide mixtures are analyzed directly by nanoLC-ESI-MSMS and after capLC separation and fractionation of peptides by MALDI-MS/MSMS, nanoESI-MSMS and/or Edman protein sequencing. MSMS peptide fragmentation data are evaluated by de novo peptide sequencing and/or protein database search using available antibody sequencing software and relevant databases. The full protein amino acid sequence is derived by putting together the peptide puzzle of overlapping peptide sequences.

Once the sequence of an antibody is known, the variable regions, such as the CDR regions, can easily by identified by state of the art bioinformatics tools known to a person of skill.

In one embodiment the present invention relates to an anti-PAD2 antibody comprising all of or a part of the sequence of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35.

The part of the sequence may comprise or consist of e.g. the variable regions of the antibody, the Fab part or one or more of the CDR regions.

In one embodiment the present invention relates to a chimeric anti-PAD2 antibody comprising at least a part of the sequence, such as a part of or all of the Fab part of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35.

In one embodiment the present invention relates to a humanized anti-PAD2 antibody comprising at least a part of the variable domain sequence of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35.

In one embodiment the present invention relates to an anti-PAD2 antibody comprising one or more of the CDR sequences, such as all of the CDR sequences of an antibody, selected from the group consisting of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35.

In one embodiment the present invention relates to an anti-PAD2 antibody comprising all of or a part of the sequence of mAb2, mAb 6 and/or mAb 34, such as all of or a part of the variable domain sequence.

In one embodiment, the present invention relates to an anti-PAD2 antibody comprising all of or a part of the sequence of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35 for use as a medicament.

In one embodiment the present invention relates to an anti-PAD2 antibody comprising all of or a part of the sequence of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35 for use in the treatment of an autoimmune disease characterized by extracellular citrullination.

In one embodiment the present invention relates to a pharmaceutical composition comprising an anti-PAD2 antibody comprising all of or a part of the sequence of mAb 1, mAb 2, mAb 3, mAb 4, mAb 5, mAb 6, mAb 7, mAb 8, mAb 9, mAb 10, mAb 11, mAb 12, mAb 13, mAb 14, mAb 15, mAb 16, mAb 17, mAb 18, mAb 19, mAb 20, mAb 21, mAb 22, mAb 23, mAb 24, mAb 25, mAb 26, mAb 27, mAb 28, mAb 29, mAb 30, mAb 31, mAb 32, mAb 33, mAb 34 and/or mAb 35 and at least one pharmaceutically acceptable diluent, carrier or excipient.

In one embodiment of the invention there is provided an anti-PAD2 antibody comprising a Heavy chain variable region (VH) selected from the group consisting of SEQ ID NO:3 (mAb#2), SEQ ID NO:13 (mAb#6) and SEQ ID NO:23 (mAb#34), or a variant thereof having at least 75% sequence identity thereto.

In one embodiment of the invention there is provided an anti-PAD2 antibody comprising a Light chain variable region (VL) selected from the group consisting of SEQ ID NO:8 (mAb#2), SEQ ID NO:18 (mAb#6) and SEQ ID NO:28 (mAb#34), or a variant thereof having at least 75% sequence identity thereto.

In one embodiment of the invention there is provided an anti-PAD2 antibody comprising a Heavy chain variable region (VH) selected from the group consisting of SEQ ID NO:3 (mAb#2), SEQ ID NO:13 (mAb#6) and SEQ ID NO:23 (mAb#34), or a variant thereof having at least 75% sequence identity thereto, and/or comprising a Light chain variable region (VL) selected from the group consisting of SEQ ID NO:8 (mAb#2), SEQ ID NO:18 (mAb#6) and SEQ ID NO:28 (mAb#34), or a variant thereof having at least 75% sequence identity thereto.

The present invention in one embodiment relates to an anti-PAD2 antibody comprising a Heavy chain variable region (VH) of sequence SEQ ID NO:3, or a variant thereof having at least 75% sequence identity thereto; and/or comprising a Light chain variable region (VL) of sequence SEQ ID NO:8, or a variant thereof having at least 75% sequence identity thereto.

The present invention in another embodiment relates to an anti-PAD2 antibody comprising a Heavy chain variable region (VH) of sequence SEQ ID NO:13, or a variant thereof having at least 75% sequence identity thereto; and/or comprising a Light chain variable region (VL) of sequence SEQ ID NO:18, or a variant thereof having at least 75% sequence identity thereto.

The present invention in yet another embodiment relates to an anti-PAD2 antibody comprising a Heavy chain variable region (VH) of sequence SEQ ID NO:23, or a variant thereof having at least 75% sequence identity thereto; and/or comprising a Light chain variable region (VL) of sequence SEQ ID NO:28, or a variant thereof having at least 75% sequence identity thereto.

In one embodiment a variant of a Light chain variable region (VL) a Heavy chain variable region (VH) having at least 75% sequence identity comprises a variant having at least 75%, such as at least 80%, for example at least 85%, such as at least 90%, for example at least 95%, such as at least 96, 97, 98 or 99% sequence identity to a Light chain variable region (VL) selected from the group consisting of SEQ ID NO:8, SEQ ID NO:18 and SEQ ID NO:28, or a Heavy chain variable region (VH) selected from the group consisting of SEQ ID NO:3, SEQ ID NO:13 and SEQ ID NO:23.

In another embodiment there is provided an anti-PAD2 antibody comprising a Heavy chain variable region (VH) comprising one, two or three binding domains selected from
1) a first binding domain comprising a VH CDR1 selected from the group consisting of SEQ ID NO:5, SEQ ID NO:15 and SEQ ID NO:25, or a sequence having at least 75% sequence identity thereto;
2) a second binding domain comprising a VH CDR2 selected from the group consisting of SEQ ID NO:6, SEQ ID NO:16 and SEQ ID NO:26, or a sequence having at least 75% sequence identity thereto; and
3) a third binding domain comprising a VH CDR3 selected from the group consisting of SEQ ID NO:7, SEQ ID NO:17 and SEQ ID NO:27, or a sequence having at least 75% sequence identity thereto;

and/or comprising

a Light chain variable region (VL) comprising one, two or three binding domains selected from
1) a first binding domain comprising a VL CDR1 selected from the group consisting of SEQ ID NO:10, SEQ ID NO:20 and SEQ ID NO:30, or a sequence having at least 75% sequence identity thereto;
2) a second binding domain comprising a VL CDR2 selected from the group consisting of SEQ ID NO:11, SEQ ID NO:21 and SEQ ID NO:31, or a sequence having at least 75% sequence identity thereto; and
3) a third binding domain comprising a VL CDR3 selected from the group consisting of SEQ ID NO:12, SEQ ID NO:22 and SEQ ID NO:32, or a sequence having at least 75% sequence identity thereto.

A binding domain as used herein denotes an antigen binding domain, which the three CDRs of each of the VH and the VL domains are mainly responsible for.

In one embodiment a sequence having at least 75% sequence identity to a CDR1, CDR2 or CDR3 sequence comprises a variant having at least 75%, such as at least 80%, for example at least 85%, such as at least 90%, for example at least 95%, such as at least 96, 97, 98 or 99% sequence identity to a VH CDR1 selected from the group consisting of SEQ ID NO:5, SEQ ID NO:15 and SEQ ID NO:25; a VH CDR2 selected from the group consisting of SEQ ID NO:6, SEQ ID NO:16 and SEQ ID NO:26, VH CDR3 selected from the group consisting of SEQ ID NO:7, SEQ ID NO:17 and SEQ ID NO:27; a VL CDR1 selected from the group consisting of SEQ ID NO:10, SEQ ID NO:20 and SEQ ID NO:30; a VL CDR2 selected from the group consisting of SEQ ID NO:11, SEQ ID NO:21 and SEQ ID NO:31; or a VL CDR3 selected from the group consisting of SEQ ID NO:12, SEQ ID NO:22 and SEQ ID NO:32.

In another embodiment there is provided an anti-PAD2 antibody comprising a Heavy chain variable region (VH) comprising one, two or three binding domains selected from
1) a first binding domain comprising a VH CDR1 selected from the group consisting of SEQ ID NO:5, SEQ ID NO:15, SEQ ID NO:25 and SEQ ID NO:34;
2) a second binding domain comprising a VH CDR2 selected from the group consisting of SEQ ID NO:6, SEQ ID NO:16 and SEQ ID NO:26; and
3) a third binding domain comprising a VH CDR3 selected from the group consisting of SEQ ID NO:7, SEQ ID NO:17, SEQ ID NO:27 and SEQ ID NO:33, and/or comprising
   a Light chain variable region (VL) comprising one, two or three binding domains selected from
1) a first binding domain comprising a VL CDR1 selected from the group consisting of SEQ ID NO:10, SEQ ID NO:20 and SEQ ID NO:30;
2) a second binding domain comprising a VL CDR2 selected from the group consisting of SEQ ID NO:11, SEQ ID NO:21 and SEQ ID NO:31; and
3) a third binding domain comprising a VL CDR3 selected from the group consisting of SEQ ID NO:12, SEQ ID NO:22, SEQ ID NO:32 and SEQ ID NO:35.

The invention also provides an anti-PAD2 antibody comprising a Heavy chain variable region (VH) comprising one, two or three binding domains selected from
1) a first binding domain comprising a VH CDR1 selected from the group consisting of SEQ ID NO:5, SEQ ID NO:15 and SEQ ID NO:25;
2) a second binding domain comprising a VH CDR2 selected from the group consisting of SEQ ID NO:6, SEQ ID NO:16 and SEQ ID NO:26; and
3) a third binding domain comprising a VH CDR3 selected from the group consisting of SEQ ID NO:7, SEQ ID NO:17 and SEQ ID NO:27.

The invention further provides an anti-PAD2 antibody comprising a Light chain variable region (VL) comprising one, two or three binding domains selected from
1) a first binding domain comprising a VL CDR1 selected from the group consisting of SEQ ID NO:10, SEQ ID NO:20 and SEQ ID NO:30;
2) a second binding domain comprising a VL CDR2 selected from the group consisting of SEQ ID NO:11, SEQ ID NO:21 and SEQ ID NO:31; and
3) a third binding domain comprising a VL CDR3 selected from the group consisting of SEQ ID NO:12, SEQ ID NO:22 and SEQ ID NO:32.

In one embodiment there is provided an anti-PAD2 antibody comprising a Heavy chain variable region (VH) comprising one, two or three binding domains selected from
1) a first binding domain comprising a VH CDR1 selected from the group consisting of SEQ ID NO:5, SEQ ID NO:15 and SEQ ID NO:25;
2) a second binding domain comprising a VH CDR2 selected from the group consisting of SEQ ID NO:6, SEQ ID NO:16 and SEQ ID NO:26; and
3) a third binding domain comprising a VH CDR3 selected from the group consisting of SEQ ID NO:7, SEQ ID NO:17 and SEQ ID NO:27, and comprising
   a Light chain variable region (VL) comprising one, two or three binding domains selected from
1) a first binding domain comprising a VL CDR1 selected from the group consisting of SEQ ID NO:10, SEQ ID NO:20 and SEQ ID NO:30;
2) a second binding domain comprising a VL CDR2 selected from the group consisting of SEQ ID NO:11, SEQ ID NO:21 and SEQ ID NO:31; and
3) a third binding domain comprising a VL CDR3 selected from the group consisting of SEQ ID NO:12, SEQ ID NO:22 and SEQ ID NO:32.

The expression comprising one, two or three binding domains selected from a first binding domain, a second binding domain and a third binding domain is to be construed as comprising either one first binding domain, or one second binding domain, or one third binding domain; or as comprising two binding domains such as a first and a second binding domain, a first and a third binding domain or a second and a third binding domain; or as comprising all three binding domains as defined herein.

In one embodiment, said anti-PAD2 antibody comprising one, two or three binding domains selected from a first binding domain, a second binding domain and a third binding domain as disclosed herein, inhibits PAD2-catalyzed citrullination of a substrate, such as fibrinogen. This may be evaluated by employing the method of Example 4.

In one embodiment, the invention relates to an anti-PAD2 antibody comprising a Heavy chain variable region (VH) comprising a VH CDR3 selected from the group consisting of SEQ ID NO:7, SEQ ID NO:17 and SEQ ID NO:27, wherein said antibody inhibits PAD2-catalyzed citrullination of a substrate such as fibrinogen.

In one embodiment, the invention provides to an anti-PAD2 antibody comprising a Heavy chain variable region (VH) comprising a VH CDR3 of sequence SEQ ID NO:33, wherein said antibody inhibits PAD2-catalyzed citrullination of a substrate such as fibrinogen.

In a particular or even preferred embodiment, the anti-PAD2 antibodies disclosed herein are humanized antibodies, such as fully humanized monoclonal antibodies, i.e. they have undergone the further process of antibody humanization of non-human monoclonal antibodies. This process is currently well-known to the skilled person, and also commercially available. For instance, software platforms which allows a robust, rapid and accurate modernized version of the traditional CDR grafting technique are developed and available, whereby the CDRs from the murine antibody sequences are identified and grafted into antibody frameworks to produce a panel of high quality, full length, humanized antibodies for expression. Antibody humanization and/or chimerization services are commercially available from i.a. Genscript (see e.g. U.S. 61/494,593), Fusion antibodies, and PX′Therapeutics.

Typical procedures for antibody humanization include one or more of steps 1-5:

1) Production and characterization of the reference murine antibody and determination of its affinity constant, e.g. by BIACORE,
2) Determination of the specific murine variable region sequences,
3) Structural modeling of the mAb variable regions and construct of a panel of variants,
4) Affinity characterization and analysis of the humanized variants followed by recombinant expression in mammalian cells, and/or
5) If required, generation of additional variants to optimize antibody affinity.

The invention is also directed to an anti-PAD2 antibody that recognizes and specifically binds to the same epitope as an anti-PAD2 antibody selected from the group consisting of an antibody comprising a VH domain identified as SEQ ID NO:3 and a VL domain identified as SEG ID NO:8 (mAb#2); an antibody comprising a VH domain identified as SEQ ID NO:13 and a VL domain identified as SEG ID NO:18 (mAb#6); and an antibody comprising a VH domain identified as SEQ ID NO:23 and a VL domain identified as SEG ID NO:28 (mAb#34).

The invention is further directed to an anti-PAD2 antibody which competes for binding to a PAD2 epitope with an anti-PAD2 antibody selected from the group consisting of an antibody comprising a VH domain identified as SEQ ID NO:3 and a VL domain identified as SEG ID NO:8 (mAb#2); an antibody comprising a VH domain identified as SEQ ID NO:13 and a VL domain identified as SEG ID NO:18 (mAb#6); and an antibody comprising a VH domain identified as SEQ ID NO:23 and a VL domain identified as SEG ID NO:28 (mAb#34).

### Therapeutic Compositions

Another aspect of the invention is a pharmaceutical composition comprising as an active ingredient an anti-PAD2 antibody, such as an anti-PAD2 antibody according to the present invention. Such compositions are intended for amelioration, prevention and/or curative treatment of autoimmune diseases characterized by extracellular citrullination, preferably hyper-citrullination. The pharmaceutical composition may be administered to a human subject or to a domestic animal or pet, but will typically be administered to humans.

In addition to at least one antibody of the invention, the pharmaceutical composition will further comprise at least one pharmaceutically acceptable diluent, carrier or excipient. These may for example include preservatives, stabilizers, surfactants/wetting agents, emulsifying agents, solubilizers, salts for regulating the osmotic pressure and/or buffers. Solutions or suspensions may further comprise viscosity-increasing substances, such as sodium carboxymethylcellulose, carboxymethylcellulose, dextran, polyvinylpyrrolidone or gelatin. A suitable pH value for the pharmaceutical composition will generally be in the range of about 5.5 to 8.5, such as about 6 to 8, e.g. about 7, maintained where appropriate by use of a buffer.

Conventional pharmaceutical practice may be employed to provide suitable formulations or compositions. The administration may be prophylactic; meaning that treatment is initiated before clinical symptoms of the disease appears. The treatment will, however, typically be therapeutic, meaning that it is administered after a particular autoimmune disease has been diagnosed due to the manifestation of clinical symptoms. Any appropriate route of administration may be employed, for example parenteral, intravenous, intra-arterial, subcutaneous, intramuscular, intraperitoneal, intranasal, aerosol, suppository or oral administration. Pharmaceutical compositions of the invention will typically be administered in the form of liquid solutions or suspensions, more typically aqueous solutions or suspensions, in particular isotonic aqueous solutions or suspensions.

The pharmaceutical compositions of the invention are prepared in a manner known per se, for example, by means of conventional dissolving, lyophilizing, mixing, granulating or confectioning processes. The pharmaceutical compositions may be formulated according to conventional pharmaceutical practice (see, for example, Remington: The Science and Practice of Pharmacy (21st edition), ed. A. R. Gennaro, 2005, Lippincott Williams & Wilkins, Philadelphia Pa. USA; Encyclopedia of Pharmaceutical Technology, ed. J. Swarbrick, 3rd edition, 2006, Informa Healthcare, New York N.Y. USA).

As an alternative to a liquid formulation, the compositions of the invention may be prepared in lyophilized form comprising the at least one antibody alone or together with a carrier, for example mannitol, in which case the composition is reconstituted with a liquid such as sterile water prior to use.

The pharmaceutical compositions in one embodiment comprise from approximately 1% to approximately 95%, preferably from approximately 20% to approximately 90%, active ingredient. Pharmaceutical compositions according to the invention may e.g. be produced in unit dose form, such as in the form of ampoules, vials, suppositories, tablets or capsules. The formulations can be administered to human individuals in therapeutically or prophylactically effective amounts (e.g., amounts which prevent, eliminate, or reduce a pathological condition) to provide therapy for an autoimmune disease or other condition. The preferred dosage of therapeutic agent to be administered is likely to depend on such variables as the severity of the disease, the overall health status of the particular patient, the formulation of the compound excipients, and its route of administration.

The antibodies and compositions of the invention will be administered in an effective amount (or suitable amount) for treatment of the condition in question, i.e. at dosages and for periods of time necessary to achieve a desired result. The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, the effect desired, and whether the anti-PAD2 antibodies are being administered as a stand-alone treatment or in combination with one or more additional treatments.

An effective amount (or suitable amount) for therapy may be measured by its ability to inhibit disease development, to stabilize disease progression and/or ameliorate symptoms in a patient, and preferably to reverse disease progression. The ability of an antibody or composition of the invention to inhibit the autoimmune disease of the present invention may be evaluated in suitable animal models that are predictive of the efficacy in human patients. Suitable dosage regimens will be selected in order to provide an optimum therapeutic response in each particular situation, for example, administered as a single bolus or as a continuous infusion, and with possible adjustment of the dosage as indicated by the exigencies of each case.

Usually a daily dosage of active ingredient can be about 0.01 to 100 milligrams per kilogram of body weight, such as about 0.1 to 80 milligrams per kilogram of body weight, for example 1 to 50 milligrams per kilogram of body weight, such as 1 to 30 milligrams per kilogram of body weight, for example 1 to 20 milligrams per kilogram of body weight, such as 1 to 10 milligrams per kilogram of body weight, for example 1 to 5 milligrams per kilogram of body weight.

The anti-PAD2 antibody may be administered once a day or several times a day. The anti-PAD2 antibody may also be administered at intervals such as once a week, twice a week, three times a week, once every other week, once every three weeks, once every four weeks or once a month.

Dosage forms suitable for administration generally contain from about 0.01 milligram to about 1000 milligrams of anti-PAD2 antibody per dose, such as about 0.05 milligram to about 500 milligrams of anti-PAD2 antibody per dose, for example about 0.1 milligram to about 500 milligrams of anti-PAD2 antibody per dose, such as about 1 milligram to about 500 milligrams of anti-PAD2 antibody per dose, for example about 5 milligram to about 500 milligrams of anti-PAD2 antibody per dose, such as about 10 milligram to about 500 milligrams of anti-PAD2 antibody per dose, for example about 20 milligram to about 500 milligrams of anti-PAD2 antibody per dose, such as about 30 milligram to about 500 milligrams of anti-PAD2 antibody per dose, for example about 40 milligram to about 500 milligrams of anti-PAD2 antibody per dose such as about 50 milligram to about 500 milligrams of anti-PAD2 antibody per dose.

In one embodiment the anti-PAD2 antibody of the present invention is administered in doses of about 0.01 to 10 mg/kg/dose, such as about 0.05 to 5.0 mg/kg/dose, for example about 0.1 to 5 mg/kg/dose, such as about 0.2 to 5.0 mg/kg/dose, for example about 0.3 to 5 mg/kg/dose, such as about 0.4 to 5.0 mg/kg/dose, for example about 0.5 to 5 mg/kg/dose.

The anti-PAD2 antibody may e.g. be administered in a similar manner as described for other biologics for treatment of RA e.g. the anti-TNF medications etanercept, adalimumab, certolizumab and golimumab are usually administered by injection under the skin (e.g. injected into the thigh or abdomen) and infliximab is administered by intravenous infusion over several hours.

In one embodiment the anti-PAD2 antibodies of the present invention are administered as a stand-alone treatment or in combination with one or more additional treatments.

In one embodiment the anti-PAD2 antibody of the present invention is administered essentially as described in the below table 2 for anti-TNF drugs for treatment of RA.

TABLE 2
Comparison of anti-TNF drugs in RA:

| | | When to expect | Methotrexate |
|---|---|---|---|
| Drug | Usual Dosing Regimen | results | needed? |
| Infliximab | Initially: Given at the clinic or at an | 2-3 weeks | Yes |
| (Remicade ®) | infusion center as an intravenous | | |
| | infusion (IV) at a dose of 3-5 mg/kg | | |
| | (according to body weight) at weeks | | |
| | 0, 2, and 6. | | |
| | Maintenance: IV infusions every | | |
| | 4-8 weeks. Dose may be increased to | | |
| | 5-10 mg/kg. | | |
| Etanercept | Initially: 50 mg once a week or 25 mg | 1-2 weeks | No |
| (Enbrel ®) | twice a week as a self-administered | | |
| | subcutaneous injection. | | |
| | Maintenance: Same | | |
| Adalimumab | Initially: 40 mg every other week as a | 2-3 weeks | Suggested, not |
| (Humira ®) | self-administered subcutaneous | | required |
| | injection. | | |
| | Maintenance: Same | | |
| Certolizumab | Initially: 400 mg on week 0, 2 and 4 | 1-2 weeks | No |
| (Cimzia ®) | as a self-administered subcutaneous | | |
| | injection. | | |
| | Maintenance: 200 mg every other | | |
| | week. | | |
| | Note: Each 400 mg dose should be | | |
| | administered as 2 injections of 200 mg | | |
| | each. | | |
| Golimumab | Initially: 50 mg once per month as a | 1-2 weeks | Yes |
| (Simponi ®) | self-administered subcutaneous | | |
| | injection. | | |
| | Maintenance: Same | | |

### Examples

The following examples are for illustrative purposes only and should not be construed as limiting the scope of the invention.

### Example 1

### Generation of PAD2 mAbs Directed Against Rabbit PAD2

### Material and Methods

### Materials

Carbazole staining solution (0.04% 3-amino-9-ethylcarbazole and 0.015% H₂O₂ in 50 mM sodium acetat buffer (50 mM CH₃COOH, 33.75 mM NaOH), pH 5.0)
Citrate/developing buffer (35 mM citric acid, 65 mM Na₂PO₄, pH 5)
Citrullination buffer (100 mM Tris-HCl, 20 mM CaCl₂, pH 7.5)
Coating buffer (15 mM Na₂CO₃, 35 mM NaHCO₃, pH 9.6)
Diluting/wash buffer (PBS+0.05% Tween 20, pH 7.3)
Elution buffer (PBS, 0.5% citric acid)
TBS buffer (0.15 M NaCl, 0.05 M Tris, pH=7.4)
Transfer-buffer (25 mM Tris, 192 mM glycine, pH 8.3, 20% ethanol)
Wash buffer (sepharose cojugating) (PBS, 0.5M NaCl)
Aluminium hydroxide, Alhydrogel 2.0% (Brenntag Biosector A/S, Frederikssund, Denmark)
Ammonium sulfate 0.2M—PEG 4000 30% solution (Sigma-Aldrich, Brøndby, Denmark)
Biotinyl-N-Hydroxy-succinimid ester (BNHS) (Sigma-Aldrich, Glostrup, Denmark)
CNBr-activated sepherose 4B (GE Healthcare, Hillerød, Denmark)
Cyanogen bromide-activated-Sepharose® 4B (Sigma-Aldrich, Brøndby, Denmark)
Freund's incomplete adjuvant (Sigma-Aldrich, Brøndby, Denmark)
HAT (Hypoxanthin-Aminopterin-Thymidin) (GIBCO® BR L, Invitrogen Corporation, Karlsruhe, Germany)
HRP conjugated polyclonal rabbit anti mouse IgG (P0260) (Dako Denmark A/S, Glostrup)
HRP conjugated streptavidin (P0397) (Dako Denmark A/S, Glostrup)
Human Fibrinogen Plasminogen Depleted, (Merck Millipore, Darmstadt, Germany)
Human recombinant PAD2 and PAD4 (tested for enzymatic activity), provided by Ger Pruijn (Dept. of Biomolecular Chemistry, NCMLS/IMM, Radboud University Nijmegen, The Netherlands).
Irrelevant mAb (culture supernatant, similar to the PAD mAbs, against chicken C3)
Irrelevant mAb 2 (culture supernatant, similar to the PAD mAbs, against SCUBE1)
Mouse anti-citrullinated fibrinogen (20B2) (Modiquest research, Nijmegen, Holland)
Novex® Sharp Pre-stained Protein Standard (Naerum, Denmark)
Novex® Sharp unstained Protein Standard (Naerum, Denmark)
Nunc MaxiSorp® flat-bottom 96 well plate (Sigma-Aldrich, Brøndby, Denmark)
Nunc-Immuno™ MicroWell™ 96 well solid plates (Sigma-Aldrich, Brøndby, Denmark)
NuPAGE, Novex, Bis-Tris Mini Gel (10% and 4-12%) (Invitrogen, CA, USA) Peptidylarginine Deiminase isolated from rabbit skeletal muscle (rPAD2)
(Sigma-Aldrich, Brøndby, Denmark)
Polyclonal rabbit anti mouse IgG (Dako Denmark A/S, Glostrup)
Polyvinylidene difluoride membranes (PVDF-HyBond, Amersham Bioscience, Uppsala, Sweden)
Precision plus protein standards—all blue stained/unstained (Bio-Rad, Copenhagen, Denmark)
RPMI-1640 (Sigma-Aldrich, Brøndby, Denmark)

### Generation of Monoclonal Antibodies

Monoclonal antibodies were raised against peptidylarginine deiminase 2 isolated from rabbit skeletal muscle (rPAD2) (Sigma-Aldrich, Brøndby, Denmark). The procedure is based on the principles described by Kohler and Milstein (Kohler and Milstein (1975) J. Immunol. 174; 2453-5).

Three BALB/c×NMRI mice were immunized subcutaneously, two times with 25 μg rPAD2 in 2 week intervals along with aluminium hydroxide (Alhydrogel 2.0%, Brenntag Biosector A/S, Frederikssund, Denmark) and Freund's incomplete adjuvant (Sigma-Aldrich, Brøndby, Denmark) in a 1:1 ratio. A small blood sample was collected from the tale of each mouse 2 weeks after second injection in order to measure titer. Serum was diluted twofold starting with a 1:1000 dilution on Nunc MaxiSorp® flat-bottom 96 well plate (MaxiSorp plates) (Sigma-Aldrich, Brøndby, Denmark) coated with 0.5 μg/mL rPAD2 and developed as described herein below. The two highest responding mice received an intravenous boost of 25 μg rPAD2 administered with Epinephrine diluted in saline water three days prior to the fusion. Hereafter, the mice generated antibody-forming B cells with specificity against the induced antigen.

The mice were killed by cervical dislocation, and their spleens were isolated and homogenized in 37° C. RPMI-1640 medium (RPMI) (Sigma-Aldrich, Brøndby, Denmark), and the homogenate was added to a final volume of 50 mL in a sterile NUNC tube. Connective tissue was removed. The Myeloma cell line (SP2/0-Ag14, a hybrid of a BALB/c spleen cell and the myeloma cell line P3X63AG8) was used as fusion partner.

The spleen cells were centrifuged at 350 g for 5 min and washed twice in serum-free RPMI medium. SP2/0-Ag14 myeloma cells (exponentially growing) were washed, centrifuged, and mixed with the spleen cells in a 1:1 ratio and the cell-mixture was centrifuged. The supernatant was removed and the tube containing the cell-pellet was kept in a 37° C. water bath for 1 min and stirred with 1.5 mL 37° C. PEG-4000 (Sigma-Aldrich, Brøndby, Denmark) to assist in the fusion of myeloma and spleen cells. The cells were then diluted at 37° C. in RPMI medium containing 10% fetal calf serum (FCS) and distributed to 20 Nunc-Immuno™ MicroWell™ 96 well solid plates (NUNC ELISA plates) (Sigma-Aldrich, Brøndby, Denmark). The plates were incubated at 37° C. with 5% CO2.

To select fused cells HAT (Hypoxanthin-Aminopterin-Thymidin) (GIBCO® BRL, Invitrogen Corporation, Karlsruhe, Germany) was added to each well. Hybridomas producing mAbs against rPAD2 or irrelevant antigens were mixed. Cell supernatant from the NUNC ELISA plates were tested for antibodies against the antigen. An antigen coating concentration of 0.2 μg/mL rPAD2 was used. Culture supernatant (100 μL) was diluted 1:1 in diluting buffer and incubated 1.5 hours at RT. After washing 3 times the plates were developed. By diluting positive wells (2-fold, 8-, 12- and 24 times), it was possible to isolate single hybridomas. Wells containing single hybridomas were detected in microscope, also with respect to cell condition, and they were transferred to NUNC culture flasks and frozen for later use—around 0.5 L was generated in 0.05% Na-azide. The procedure was repeated until all positive hybridomas were single-cell isolated.

The mAbs were purified from the culture supernatant using protein-A affinity chromatography on an Äkta FPLC system (Amersham Pharmacia, San Fransisco, Calif.) according to manufactures recommendations. The mAbs were dialyzed in PBS and stored at 4° C. with azid.

### ELISA

The procedures described in the following are general Enzyme Linked Immuno Sorbent Assay (ELISA) protocols. The assays were optimized for every single assay to give highest possible signal and still preventing unspecific signals (background). The specific conditions are stated in figure legends.

Antigen or antibodies were immobilized on MaxiSorp plates in coating buffer (Na2CO3, 35 mM NaHCO3, pH 9.6) 0/N at 4° C. The coated MaxiSorp plates were washed 3 times- and blocked in wash buffer/diluting buffer (PBS+0.05% Tween 20, pH 7.3) for at least 15 min. Tween 20 blocks unoccupied binding sites on the polystyrene surface to prevent binding of secondary reactants to the plates.

Complex-bound mAbs were detected using secondary horseradish peroxidase (HRP) conjugated reactants.
HRP-conjugated streptavidin (Dako Denmark A/S, Glostrup) was used to detect biotin labeled mAbs i.e. sandwich ELISA.
HRP-conjugated polyclonal rabbit anti mouse IgG (Dako Denmark A/S, Glostrup) were used to detect non labeled mAbs i.e. in detecting positive wells, indirect ELISA and titer determination.

To detect HRP-bound reactants, 100 μL of o-phenylene-diamine (0.4 mg/mL; Kem-En-Tec, Taastrup, Denmark) in substrate buffer (OPD substrate) (35 mM citric acid, 67 mM Na2HPO4, 0.012% H2O2, pH 5.0) was added, and 15 min later the color development was stopped by the addition of 1 M H2SO4. Optical density (OD) was measured at 490-650 nm using Vmax Kinetic Microplate Reader and the data were processed using SoftMax Pro software (Molecular Devices, Wokingham, United Kingdom).

### Indirect ELISA

The following method was used for screening and sub-cloning of the hybridomas, titer determinations and for different experiments to test the affinity of the isolated mAbs. Plates were coated with antigen as described above. Plates were incubated for 1.5 hour at RT on an orbitshaker with culture supernatants diluted 1:1 in diluting buffer followed by incubation with HRP rabbit anti mouse IgG for 45 min. Plates were developed as stated above. Plates were washed three times in between each step.

### SDS-PAGE

NuPAGE, Novex, Bis-Tris Mini Gels (10% and 4-12%) (Invitrogen, CA, USA) were used according to the manufacturer's recommendations. Samples were reduced with DDT or added non-reduced, boiled for 2 min to assist in protein denaturing, and loaded to the wells with SDS sample buffer, including a size marker. Gels containing separated proteins could be further analyzed in western blotting.

### Western Blotting

Different samples were loaded in gels (same procedure as SDS-PAGE) along with a pre-stained marker. To make the proteins accessible for the antibodies, the proteins were electro-blotted from the gel onto polyvinylidene difluoride membranes (PVDF-HyBond, Amersham Bioscience, Uppsala, Sweden), using tank blotting in transfer-buffer (25 mM Tris, 192 mM glycine, pH 8.3, 20% ethanol) with a current of 0.8 mA/cm2. The membrane was blocked in wash buffer and sliced into strips. Antibody culture supernatant (2 mL) was added 1:1 in diluting buffer and incubated 0/N at 4° C. HRP-conjugated rabbit anti mouse IgG was added 1:3000 followed by incubation 1 h/RT. Finally, the strips were washed, incubated 10 min in acetate buffer (50 mM CH3COOH, 33.75 mM NaOH, pH 5.0) and stained in carbazole staining solution (0.04% 3-amino-9-ethylcarbazole and 0.015% H2O2) in acetate buffer. Antibodies with affinity against the target protein were observed as red bands, and the approximate size could be held against the marker loaded on the gel.

### Results

### Isolation of Hybridomas

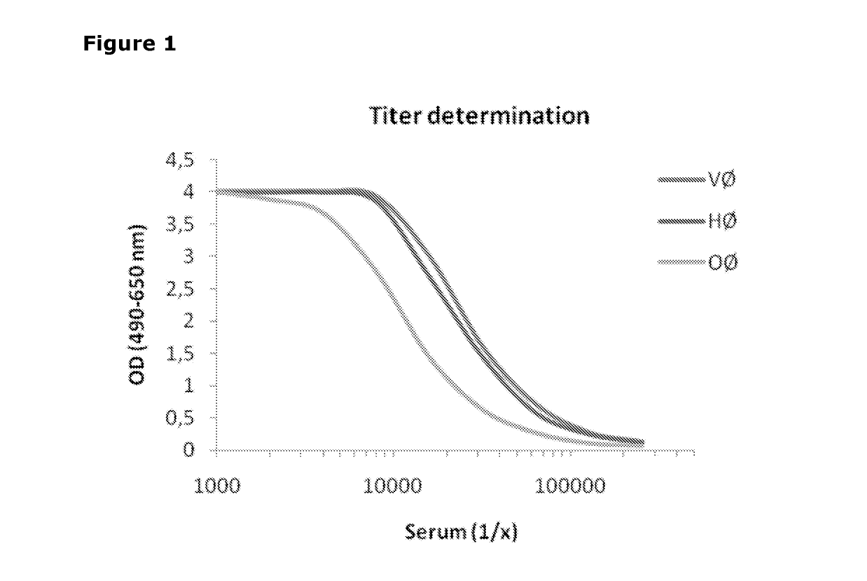

The anti-PAD titer was measured on blood samples from the three immunized mice prior to the fusion (FIG. 1). The mice “VØ” and “HØ” presented the highest antibody response against rPAD2 and were therefore selected for hybridoma formation. The titer was above 1:16,000 for both mice, which indicated a significant antibody production against rPAD2. After selection of fused cells, the culture supernatants from each well were screened with respect to content of antibody against rPAD2 in indirect ELISA. Cells from positive wells were sub-cloned in new ELISA plates. Around 100 wells were sub-cloned from the original 20 ELISA plates. Positive wells were sub-cloned until positive culture supernatants were formed by single clones. It was possible to isolate single clones following 4-10 sub-clonings.

From the screening and sub-cloning procedure, 35 hybridomas were successfully isolated. mAbs from the individual isolated hybridomas are referred to as mAb #1-35. mAb #1-35 were characterized further in order to select individual mAbs to be exploited in various experiments.

Characterization of mAbs Against rPAD2

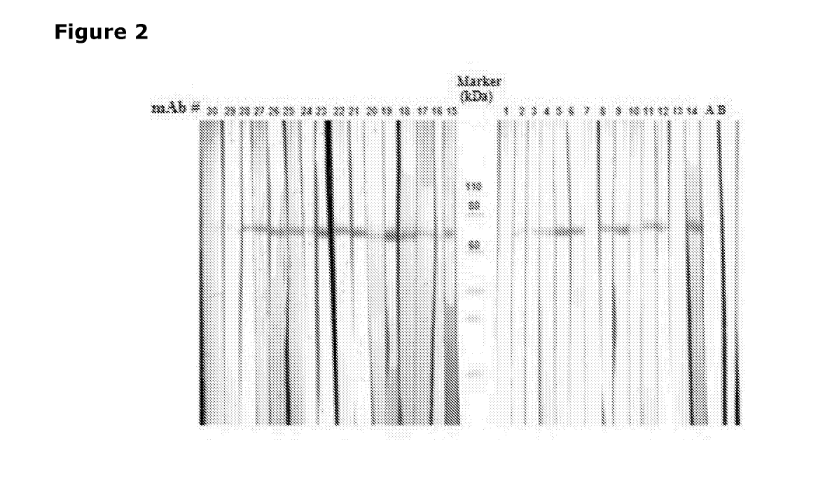

All mAbs were tested by western blotting to confirm the reactivity against rPAD2 (FIG. 2 and table 3).

TABLE 3
mAbs grouped with respect to reactivity in western
blotting against rPAD2.

| Negative mAbs | mAb#: 13 (including controls A and B) |
|---|---|
| Weakly reacting mAbs | mAb#: 1, 2, 3, 4, 7, 10, 16, 17, 20, 24, 29, 30 |
| Strongly reacting mAbs | mAb#: 5, 6, 8, 9, 11, 12, 14, 15, |
| | 18, 19, 21, 22, 23, 25, 26, 27, 28 |

All of the mAbs, except mAb #13, showed reactivity with rPAD2 in western blotting. Some mAbs reacted but weakly, e.g. mAb #1, 7 and 30, whereas others gave a strong significant band, e.g. mAb #5, 6 and 9. The controls were negative indicating that no secondary reagents or anything despite anti-PAD mAbs in the culture supernatants were responsible for the signals. mAb #31-35 were not isolated at the time of this experiment.

### Discussion

rPAD2 was used in the present study for immunization of mice and generation of monoclonal antibodies. Rabbit PAD2 and human PAD2 have 94% sequence identity. The titer of the blood, from the immunized mice, was measured prior to the fusion to test the antibody response against the induced antigen. Titer was in this respect defined as the reciprocal dilution where the absorbance is 50% of the maximum signal. This titer was higher than 16,000, which is, by our experience, necessary for a successful fusion process.

By screening and sub-cloning hybridomas, it was possible to isolate 35 single clones with reactivity against rPAD2. A coating concentration of 0.2 μg/mL rPAD2 was used for this procedure, securing that only mAbs detecting rPAD2 at this concentration and below were selected. The generated mAbs are high-affinity mAbs.

The hybridomas can lose their ability to produce antibodies during the screening/sub-cloning procedure. After sub-cloning and proliferation they can lose chromosomes, including the loci responsible for antibody production. After sub-cloning 3-4 times they seemed to have been stabilized. It was possible to isolate single hybridomas during 4-10 sub-clonings.

All isolated mAbs bound rPAD2 in an indirect ELISA—the procedure used for screening and isolation of mAb #1-35. The reactivity against rPAD2 was confirmed by western blotting (FIG. 2). The western blot also visualized the target of the mAbs with respect to size. The bands between 60- and 80 kDa correspond to the theoretical size of rPAD2. Not all mAbs detected rPAD2 well using this technique. Blotted proteins are denatured, and thereby antigenically different from the native induced form. mAbs recognizing linear epitopes are best suited for PAD recognition using this technique.

### Example 2

### mAbs Cross-Reacting with Human PAD2

Materials and methods as described in example 1.

### Results

In order to identify mAbs which cross-react with human PAD2 (hPAD2), different experiments were carried out (ELISA and western blotting). FIG. 5 summarizes the results obtained from the different experiments regarding human recombinant PAD2-detection and indicates which of the mAbs that bind human PAD2.

### Indirect ELISA

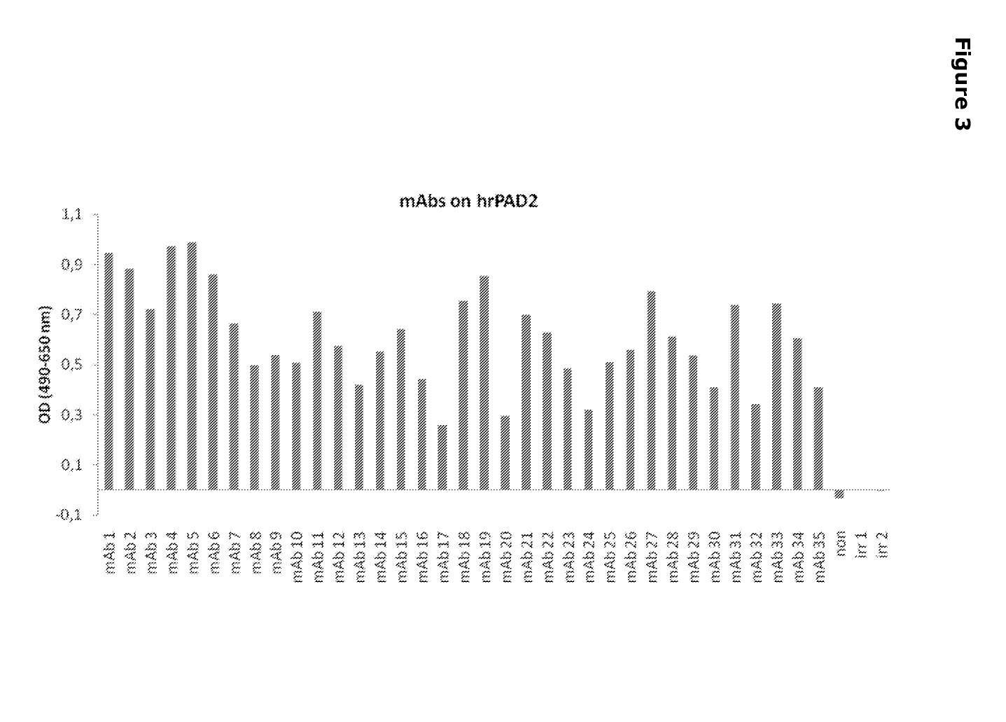

An indirect ELISA was performed to test if the mAbs could be used to detect human recombinant PAD2 (hrPAD2) in ELISA. All culture supernatants were tested against hrPAD2 in an affinity ELISA (FIG. 3).

All 35 mAbs recognized hrPAD2 in this experiment. The data from all dilutions can be seen in Table 4 below.

TABLE 4
ELISA dilutions

| | coat-konc. (ng) | | | | | | | | | | | |
|---|---|---|---|---|---|---|---|---|---|---|---|---|
| | 500 | 250 | 125 | 62.5 | 32.25 | 15.6 | 7.8 | 3.9 | 2 | 0.97 | 0.48 | 0 |
| mAb 1 | 2.352 | 2.164 | 1.874 | 1.429 | 0.946 | 0.571 | 0.305 | 0.162 | 0.073 | 0.034 | 0.017 | 0.044 |
| mAb 2 | 2.302 | 2.101 | 1.789 | 1.37 | 0.882 | 0.513 | 0.261 | 0.127 | 0.056 | 0.018 | −0.002 | 0.052 |
| mAb 3 | 2.303 | 2.088 | 1.738 | 1.219 | 0.723 | 0.389 | 0.171 | 0.073 | 0.028 | 0.002 | −0.008 | 0.06 |
| mAb 4 | 2.383 | 2.202 | 1.894 | 1.471 | 0.972 | 0.585 | 0.301 | 0.16 | 0.08 | 0.037 | 0.016 | 0.062 |
| mAb 5 | 2.418 | 2.212 | 1.94 | 1.471 | 0.987 | 0.594 | 0.327 | 0.171 | 0.089 | 0.043 | 0.025 | 0.037 |
| mAb 6 | 2.405 | 2.199 | 1.827 | 1.378 | 0.861 | 0.469 | 0.237 | 0.117 | 0.062 | 0.028 | 0.014 | 0.035 |
| mAb 7 | 2.395 | 2.133 | 1.738 | 1.169 | 0.665 | 0.373 | 0.17 | 0.075 | 0.035 | 0.017 | 0.006 | 0.038 |
| mAb 8 | 1.993 | 1.804 | 1.442 | 0.957 | 0.498 | 0.273 | 0.09 | 0.021 | −0.001 | −0.013 | −0.002 | 0.039 |
| mAb 9 | 2.324 | 2.005 | 1.588 | 1.024 | 0.539 | 0.269 | 0.123 | 0.052 | 0.031 | 0.02 | 0.001 | 0.034 |
| mAb | 2.304 | 1.913 | 1.546 | 1.027 | 0.507 | 0.414 | 0.357 | 0.188 | 0.075 | 0.031 | −0.022 | 0.058 |
| 10 | | | | | | | | | | | | |
| mAb | 2.355 | 2.076 | 1.717 | 1.246 | 0.713 | 0.647 | 0.527 | 0.285 | 0.041 | 0.038 | 0.009 | 0.053 |
| 11 | | | | | | | | | | | | |
| mAb | 2.352 | 2.061 | 1.651 | 1.102 | 0.574 | 0.241 | 0.196 | 0.093 | 0.043 | 0.023 | 0.01 | 0.038 |
| 12 | | | | | | | | | | | | |
| mAb | 2.235 | 1.885 | 1.451 | 0.876 | 0.42 | 0.19 | 0.081 | 0.04 | 0.02 | 0.129 | 0.025 | 0.09 |
| 13 | | | | | | | | | | | | |
| mAb | 2.333 | 2.016 | 1.618 | 1.039 | 0.552 | 0.264 | 0.123 | 0.058 | 0.134 | 0.129 | 0.059 | 0.035 |
| 14 | | | | | | | | | | | | |
| mAb | 2.304 | 2.006 | 1.675 | 1.124 | 0.643 | 0.301 | 0.182 | 0.099 | 0.054 | 0.032 | 0.021 | 0.077 |
| 15 | | | | | | | | | | | | |
| mAb | 1.881 | 1.563 | 1.16 | 0.515 | 0.442 | 0.212 | 0.088 | 0.018 | 0.037 | 0.012 | 0.014 | 0.515 |
| 16 | | | | | | | | | | | | |
| mAb | 2.331 | 1.927 | 1.31 | 0.665 | 0.258 | 0.117 | 0.049 | 0.025 | 0.011 | 0.008 | 0.013 | 0.04 |
| 17 | | | | | | | | | | | | |
| mAb | 2.385 | 2.094 | 1.787 | 1.312 | 0.754 | 0.419 | 0.195 | 0.099 | 0.044 | 0.018 | 0.012 | 0.05 |
| 18 | | | | | | | | | | | | |
| mAb | 2.429 | 2.145 | 1.83 | 1.337 | 0.853 | 0.464 | 0.255 | 0.118 | 0.056 | 0.029 | 0.027 | 0.051 |
| 19 | | | | | | | | | | | | |
| mAb | 1.356 | 1.158 | 0.865 | 0.617 | 0.295 | 0.147 | 0.058 | 0.015 | 0.013 | 0.068 | −0.007 | 1.106 |
| 20 | | | | | | | | | | | | |
| mAb | 2.34 | 2.069 | 1.76 | 1.22 | 0.701 | 0.382 | 0.178 | 0.104 | 0.052 | 0.026 | 0.011 | 0.078 |
| 21 | | | | | | | | | | | | |
| mAb | 2.374 | 2.001 | 1.719 | 1.167 | 0.63 | 0.322 | 0.155 | 0.081 | 0.06 | 0.017 | 0.008 | 0.044 |
| 22 | | | | | | | | | | | | |
| mAb | 2.287 | 1.857 | 1.5 | 0.92 | 0.485 | 0.255 | 0.126 | 0.055 | 0.029 | 0.009 | −0.013 | 0.058 |
| 23 | | | | | | | | | | | | |
| mAb | 2.322 | 1.923 | 1.365 | 0.749 | 0.322 | 0.142 | 0.062 | 0.028 | 0.019 | 0.007 | 0.002 | 0.036 |
| 24 | | | | | | | | | | | | |
| mAb | 2.47 | 2.104 | 1.554 | 0.978 | 0.509 | 0.235 | 0.1 | 0.044 | 0.016 | 0.006 | −0.001 | 0.047 |
| 25 | | | | | | | | | | | | |
| mAb | 2.515 | 2.103 | 1.688 | 1.056 | 0.557 | 0.254 | 0.107 | 0.044 | 0.012 | −0.005 | 0.005 | 0.07 |
| 26 | | | | | | | | | | | | |
| mAb | 2.47 | 2.236 | 1.801 | 1.282 | 0.794 | 0.451 | 0.235 | 0.114 | 0.059 | 0.025 | 0.009 | 0.049 |
| 27 | | | | | | | | | | | | |
| mAb | 2.477 | 2.132 | 1.659 | 1.11 | 0.612 | 0.313 | 0.156 | 0.076 | 0.038 | 0.021 | 0.011 | 0.034 |
| 28 | | | | | | | | | | | | |
| mAb | 2.415 | 2.091 | 1.616 | 1.045 | 0.535 | 0.245 | 0.1 | 0.067 | 0.029 | 0.013 | 0.004 | 0.035 |
| 29 | | | | | | | | | | | | |
| mAb | 2.427 | 1.967 | 1.473 | 0.849 | 0.412 | 0.176 | 0.076 | 0.039 | 0.019 | 0.008 | 0.003 | 0.036 |
| 30 | | | | | | | | | | | | |
| mAb | 2.465 | 2.158 | 1.819 | 1.234 | 0.738 | 0.405 | 0.199 | 0.095 | 0.048 | 0.038 | 0.012 | 0.049 |
| 31 | | | | | | | | | | | | |
| mAb | 2.403 | 1.974 | 1.377 | 0.709 | 0.344 | 0.168 | 0.062 | 0.029 | 0.013 | 0.001 | −0.002 | 0.044 |
| 32 | | | | | | | | | | | | |
| mAb | 2.415 | 2.165 | 1.716 | 1.204 | 0.745 | 0.428 | 0.221 | 0.115 | 0.065 | 0.034 | 0.02 | 0.043 |
| 33 | | | | | | | | | | | | |
| mAb | 2.377 | 2.073 | 1.592 | 1.03 | 0.607 | 0.326 | 0.168 | 0.081 | 0.042 | 0.023 | 0.023 | 0.039 |
| 34 | | | | | | | | | | | | |
| mAb | 2.225 | 1.809 | 1.265 | 0.726 | 0.411 | 0.205 | 0.095 | 0.051 | 0.024 | 0.017 | 0.022 | 0.044 |
| 35 | | | | | | | | | | | | |
| non | −0.027 | −0.029 | −0.032 | −0.022 | −0.031 | −0.032 | −0.028 | −0.032 | −0.028 | −0.026 | −0.031 | 0.067 |
| irr 1 | 0.006 | 0.001 | 0.002 | 0.004 | 0.003 | 0.001 | 0 | 0.005 | 0.001 | 0.001 | −0.001 | 0.035 |
| irr 2 | 0.005 | 0.002 | 0.003 | −0.001 | −0.002 | 0 | 0.002 | 0.003 | 0.001 | 0 | 0.004 | 0.035 |

Culture supernatants from mAb #1-35 tested on human PAD2 coated plates.
Irr1 (culture supernatant) is anti-SCUBE1.
hPAd2 was 2-fold diluted from 500 ng/mL.
HRP Rabbit anti-mouse (p0260) was added 1:1000 for 1 hour at RT and plates were developed with OPD substrate. The levels are given as OD₄₉₀₋₆₅₀ nm-units.

FIG. 3 shows all mAbs compared with regard to absorbance at a coating concentration of 32 ng/mL.

### Western Blotting

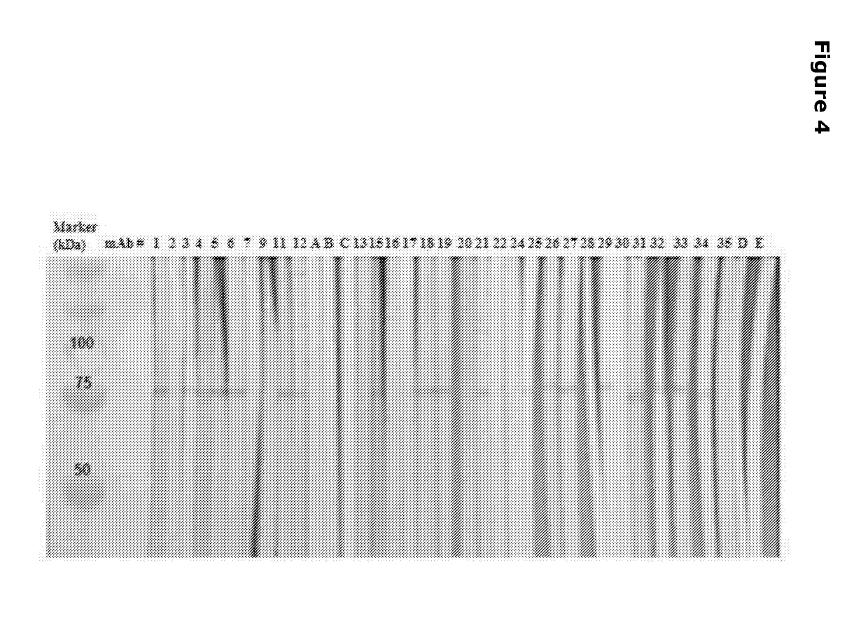

The mAbs were further tested by western blotting against hrPAD2 (FIG. 4).

Several of the mAbs showed a significant band at around 75 kDa, which indicates specific binding. The negative controls showed no signal, and a number of mAbs were negative as well. Table 5 shows which mAbs were negative, weakly reacting or strongly reacting.

TABLE 5
mAbs tested in western blotting with hrPAD2.

| Negative mAbs | mAb#: 7, 13, 16, 20, 22, 24, 28, 30 |
|---|---|
| Weakly reacting mAbs | mAb#: 2, 4, 9, 15, 17, 26, 32, 35 |
| Strongly reacting mAbs | mAb#: 1, 3, 5, 6, 11, 12, 18, 19, 21, 25, |
| | 27, 29, 30, 31, 33, 34 |

A summary of the results arising from the ELISA and western blotting experiments are shown in FIG. 5.

An indirect ELISA was performed to see if any of the mAbs cross-reacted with human recombinant PAD4 (FIG. 6). None of the mAbs bound hrPAD4 and all mAbs seem to be PAD2 specific.

Both hrPAD2 and hrPAD4 were functionally active.

### Discussion

Several of the generated mAbs were found to react well with human PAD2 in ELISA and western blotting analyses. None of the mAbs reacted with human PAD4 when tested in indirect ELISA, showing the specificity of the mAbs for PAD2. The amino acid conservation among PAD isoforms is between 50-55%, however according to the present study, the generated mAbs seem to specifically recognise the PAD2 isoform.

The basic idea for therapeutic usage of anti-PAD2 mAbs in RA patients is to inhibit PAD2-catalyzed citrullination and thereby generation of autoantigens (ACPAs) for B- and T cells. Intracellular citrullination is important for several processes. A major advantage of using mAbs directed against PAD2 rather than chemical (small molecule) inhibitors is that antibodies target and inhibit extracellular citrullination specifically, thus preserving vital functions of intracellular citrullination.

As ACPA's can be detected years before the onset of clinical disease, antibody-mediated inhibition of PAD2 may represent a treatment proximal to conventional mAb treatments of RA, i.e. neutralisation of cytokines such as TNF-alpha, IL-1-beta, or IL-6. Neutralisation of these cytokines can cause serious adverse effects, including lymphomas and other cancers, reactivation of latent infections such as tuberculosis, or viral infections. Such serious side-effects are not expected from blockade of PAD2 activity.

### Example 3

### Epitope Mapping of Anti-PAD2

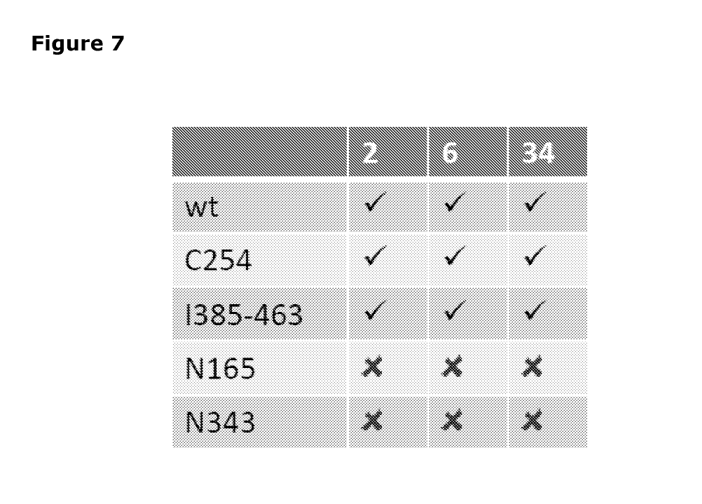

In order to identify the epitope of human PAD2 (hPAD2), different experiments were carried out. FIG. 7 summarizes the results obtained and indicates that the epitope of human PAD2 is to be found in the N-terminal part of the protein, more specifically within amino acids 1-165 (since the mAbs tested do not bind the N165 splice variant). As the epitope (often comprising 8-10 aa's) may stretch over aa position 165, being incomplete due to the splicing, the epitope is at least within aa's 1-175.

Different splice variants of PAD2 were evaluated by western blotting. Shown is the reactivity of three selected mAbs (#2, #6 and #34) with VVT (full length wild type human PAD2), C254 (amino acids 1-254 of human PAD2), 1385-463 (whole length human PAD2 without the catalytic site), N165 (from amino acid 165 to the C-terminus), N343 (from amino acid 343 to the C-terminus). The mAbs were found to bind in the N-terminal region among amino acids 1-165.

### Example 4

### Evaluation of Inhibitory Capacity of Anti-PAD2 mAbs

Experiments were carried out in order to evaluate the specific inhibitory activity of the anti-PAD2 antibodies of the present invention towards PAD2 activity.

The ability of selected anti-PAD2 mAbs to inhibit citrullination of fibrinogen was tested using human recombinant PAD2 (hrPAD2) as catalyst. Fibrinogen (2 μ/mL) was immobilized to ELISA plates, and anti-PAD2 mAbs was applied along with hrPAD2. Citrullination of fibrinogen was detected using mouse anti-citrullinated fibrinogen (20B2) from Modiquest research. (A) Test of the inhibitory capacity of mAbs #2, #3 and #33. A mAb against human complement component 4 (C4) was used as negative control. (B) Test of the inhibitory capacity of mAbs #6, #8 and #10. Anti-C4 was used as control. (C) Test of the inhibitory capacity of culture supernatants (cs) from mAb #9, #12, #31 and #34 was tested. mAbs against chicken complement component 3 (chC3) and SCUBE1 (signal peptide, CUB domain, epidermal growth factor-like protein 1) was used as controls. The mAbs performing better in this assay were mAb #2 (A), 6 (B) and 34 (C).

### Example 5

### DNA Sequencing of Anti-PAD2 Antibodies

The objective is to perform the following analysis for the samples:

mAb#2; Mouse IgG monoclonal antibody, unknown subtype
mAb#6; Mouse IgG monoclonal antibody, unknown subtype
mAb#34; Mouse IgG monoclonal antibody, unknown subtype
clone 2 11-26-2; Mouse hybridoma cells for mAb#2
clone 6 11-26-6; Mouse hybridoma cells for mAb#6
clone 2 11-26-34; Mouse hybridoma cells for mAb#34
DNA sequencing of HC and LC variable regions
High coverage peptide map, 1 protease (Trypsin, LC-MSMS) by database searching:
   1) against the variable regions obtained by DNA sequencing, and
   2) against public known mouse IgG sequences for identification of constant sequences

### Summary and Conclusion:

The DNA sequencing was successful and provided 1 heavy and 1 light chain variable region for each hybridoma. The mass spectrometric peptide mapping by trypsin digestion, LC MS/MS analysis and Mascot database searching confirmed the proposed protein sequences. The database search against the NCBI protein database found Mouse IgG antibodies for all 6 chains. Proposed full length sequences are provided by stitching together the DNA variable sequence with the best match database constant region. The peptide maps against the Stitched sequences are also provided in the report.

### Materials and Methods:

### Cloning and Sequencing Hybridoma Antibody Heavy and Light Chain Variable Regions

The hybridomas had been harvested from a 75 cm2 cell growth plate with confluent cell density and frozen in Fetal Calf Serum+10% DMSO. The cells were thawed at Alphalyse and 1 mL cells were transferred to a 15 mL centrifuge tube containing 5 mL RNAprotect Cell Reagent (Qiagen #76526). Estimated number of cells 0.5×10 7. The vials were frozen at −20 C, and shipped to Syd Labs for DNA sequencing.

### Methods for RACE Identification of Heavy and Light Chains

RACE (Rapid Amplification of cDNA Ends) was performed using Clontech SMARTer
RACE cDNA Amplification Kit (Cat. No. 634923), according to steps 1-9 below:
1. Total RNA extraction

Total RNA was extracted from hybridoma cells using QIAGEN® RNeasy Mini Kit (Cat No. 74104).

2. mRNA denaturing
mRNA Mix:

| | | |
|---|---|---|
| RNA template (0.2-0.4 ug) | 1-2.75 | μl |
| 5′-RACE Primer A | 1 | μl |
| RNase-free water | to 3.75 | μl |

Incubate the mRNA Mix at 72° C. for 3 min, then cool the tubes to 42° C. for 2 min. After cooling, spin the tubes briefly for 10 seconds at 14,000×g to collect the contents at the bottom.

3. cDNA synthesis

### Reaction Setup:

| | |
|---|---|
| SMARTer II A Oligo | 1 μl |
| 5X First-Strand Buffer | 2 μl |
| DTT (20 mM) | 1 μl |
| dNTP Mix (10 mM) | 1 μl |
| RNase Inhibitor | 0.25 μl |
| SMARTScribe RT | 1 μl |
| mRNA Mix after denaturing | 3.75 μl |
| Total volume | 10.0 μl |

Incubate the tubes at 42° C. for 90 min, heat tubes at 70° C. for 10 min. Dilute cDNA in 20 μl Tricine-EDTA buffer and store at −20° C.

### 4. 5′ RACE Reaction

### Reaction Setup:

| | |
|---|---|
| 2X PCR mix | 10 μl |
| cDNA from Step 3 | 1 μl |
| 10X Universal Primer Mix | 2 μl |
| Reverse primer | 1 μl |
| RNase-free water | 6 μl |
| Total volume | 20.0 μl |

PCR conditions:
Initial denaturing: 95° C., 5 min
25 cycles of 95° C., 25 sec
60° C., 30 sec
68° C., 30 sec
   Final extension: 68° C., 10 min
   5. Analyzing PCR results

After PCR is finished, PCR reaction samples were analyzed on an agarose gel to visualize the amplified DNA fragments. The correct antibody variable region DNA fragments should have a size between 500-1000 base pairs.

### 6. TOPO Cloned PCR Positive Bands

7. PCR-amplified TOPO clones

Followed by gel electrophoresis and recovery from agarose gel

8. Sequencing Approximately 10 clones in total.
9. CDR analysis

Performed CDR analysis using sequencing data (CDR regions were defined using VBASE2, http://www.vbase2.org/)

### High Coverage Peptide Map by LC-MS/MS Analysis and Database Search

The protein sample was denatured in GndHCL buffer, reduced and alkylated with iodoacetamide, i.e. carbamidomethylated, and subsequently digested with trypsin that cleaves after lysine and arginine residues. The resulting peptides were injected on an Agilent 1200 HPLC system connected to a QTOF mass spectrometer (Maxis Impact from Bruker). The MS/MS spectra were used for Mascot database searching. The data are searched against an in-house custom database containing the specific protein sequences obtained by DNA sequencing of the variable domains of the hybridomas provided by the client. The data were also searched against the nrdb protein sequence database downloaded from NCBI, for matching of the constant regions.

The Mascot software finds matching proteins in the database by their peptide masses and peptide fragment masses. The protein identification is based on a probability-scoring algorithm (www.matrixscience.com) and the significant best matching protein is shown in the result report. Homologous proteins with a lower score are not included in the report.

The identified database protein sequences are shown in the Results together with the obtained mass spectrometric peptide maps. The peptides used for the identification are highlighted in the sequence and the matching peptides are listed for comparison of the determined and calculated values.

Results—mAb#2 Sequences:

Heavy chain variable region (VH): amino acid sequence (SEQ ID NO:3)

QVQLQQPGAELVRPGASVKLSCKASGYTFTSYWMNWVKQRPGQGLEWI
GMIDPSDSESYYNQMFKDKATLTVDKSSSTAYMQLSRLTSEDSAVYYCA
RKDYYAYGGAMDYWGQGTSVTVSS

Heavy chain variable region (VH): nucleotide sequence (SEQ ID NO:4)

CAGGTCCAACTGCAGCAGCCTGGGGCTGAACTGGTGAGGCCTGGGGCTT
CAGTGAAGCTGTCCTGCAAGGCTTCTGGCTACACCTTCACCAGCTACTGG
ATGAACTGGGTGAAGCAGAGGCCTGGACAAGGCCTTGAATGGATTGGTAT
GATTGATCCTTCAGACAGTGAAAGTTATTACAATCAAATGTTCAAGGACA
AGGCCACATTGACTGTAGACAAATCCTCCAGCACAGCCTACATGCAGCTC
AGCAGGTTGACATCTGAGGACTCTGCGGTCTATTACTGTGCAAGAAAGGA
TTACTACGCCTACGGGGGAGCTATGGACTACTGGGGTCAAGGAACCTCAG
TCACCGTCTCCTCA

Heavy chain variable region (VH)—CDR analysis:

----CDR1---> <--CDR2--> <---CDR3----
GYTFTSYW.... IDPSDSES.. ARKDYYAYGGAMDY
CDR1 (VH):

(SEQ ID NO: 5)

GYTFTSYW
CDR2 (VH):

(SEQ ID NO: 6)

IDPSDSES
CDR3 (VH):

(SEQ ID NO: 7)

ARKDYYAYGGAMDY

Light chain variable region (VL): amino acid sequence (SEQ ID NO:8)

QIVLTQSPAILSASLGEEITLTCSANSSARYMHWYQQKSGTSPKLLIY
STSNLASGVPSRFSGSGSGTFYSLTLSSVEAEDAADYYCHQWSGY
PTFGGGTKLEIKR

Light chain variable region (VL): nucleotide sequence (SEQ ID NO:9)

CAGATTGTTCTCACCCAGTCTCCAGCAATCCTGTCTGCATCTCTAGGGG
AGGAGATCACCCTAACCTGCAGTGCCAACTCGAGTGCACGTTACATGC
ACTGGTACCAGCAGAAGTCAGGCACTTCTCCCAAACTCTTGATTTATA
GCACATCCAACCTGGCTTCTGGAGTCCCTTCTCGCTTCAGTGGCAGT
GGGTCTGGGACCTTTTATTCTCTCACACTCAGCAGTGTGGAGGCTGA
AGATGCTGCCGATTATTACTGCCATCAGTGGAGTGGTTATCCCACGT
TCGGAGGGGGGACCAAGCTGGAAATAAAACG

Light chain variable region (VL)—CDR analysis:

----CDR1---> <--CDR2--> <---CDR3----
SSARY....... STS....... HQWSGYPT
CDR1 (VL):

(SEQ ID NO: 10)

SSARY
CDR2 (VL):

(SEQ ID NO: 11)

STS
CDR3 (VL):

(SEQ ID NO: 12)

HQWSGYPT

Results—mAb#6 Sequences:

Heavy chain variable region (VH): amino acid sequence (SEQ ID NO:13)

EVQLQQSGPDLVKPGASVKISCKASGYSFTAYYMHWVKQSHGESLEWIG
RVNPNNGGSSYNQKFKGKAILTVHKSSNTAYMELRSLTSEDSAVYFCAR
GDYLPSLGYWGQGTSVTVSS

Heavy chain variable region (VH): nucleotide sequence (SEQ ID NO:14)

GAGGTCCAGCTGCAGCAGTCTGGACCTGACCTGGTGAAGCCTGGGGCTT
CAGTGAAGATATCCTGCAAGGCTTCTGGTTACTCATTCACTGCCTACTA
CATGCACTGGGTGAAGCAGAGCCATGGAGAGAGCCTTGAGTGGATTGGA
CGTGTTAATCCTAACAATGGTGGTAGTAGCTACAACCAGAAGTTCAAGG
GCAAGGCCATATTAACTGTACACAAGTCATCCAACACAGCCTACATGGA
GCTCCGCAGCCTGACATCTGAGGACTCTGCGGTCTATTTCTGTGCAAGG
GGGGATTACCTTCCCTCTTTGGGCTACTGGGGTCAAGGAACCTCAGTCA
CCGTCTCCTCA

Heavy chain variable region (VH)—CDR analysis:

----CDR1---> <--CDR2--> <---CDR3----
GYSFTAYY.... VNPNNGGS.. ARGDYLPSLGY
CDR1 (VH):

(SEQ ID NO: 15)

GYSFTAYY
CDR2 (VH):

(SEQ ID NO: 16)

VNPNNGGS
CDR3 (VH):

(SEQ ID NO: 17)

ARGDYLPSLGY

Light chain variable region (VL): amino acid sequence (SEQ ID NO:18)

DIVMTQSQKFMSTSVGDRVSVTCKASQNVDTTVAWYQQKPGQSPKALIY
SASYRYSGVPDRFTGSGSGTDFTLTVTNVQSEDLAEYFCQQYDSYPFTF
GSGTKLEIK

Light chain variable region (VL): amino acid sequence (SEQ ID NO:19)

GACATTGTGATGACCCAGTCTCAAAAATTCATGTCCACATCAGTAGGAG
ACAGGGTCAGCGTCACCTGCAAGGCCAGTCAGAATGTGGATACTACTGT
AGCCTGGTATCAACAGAAACCAGGACAATCTCCTAAAGCACTGATTTAC
TCGGCATCCTACCGGTACAGTGGAGTCCCTGATCGCTTCACAGGCAGTG
GATCTGGGACAGATTTCACTCTCACCGTCACCAATGTGCAGTCTGAAGA
CTTGGCAGAGTATTTCTGTCAGCAATATGACAGCTATCCATTCACGTTC
GGCTCGGGGACAAAGTTGGAAATAAAAC

Light chain variable region (VL)—CDR analysis:

----CDR1---> <--CDR2--> <---CDR3----
QNVDTT...... SAS....... QQYDSYPFT
CDR1 (VL):

(SEQ ID NO: 20)

QNVDTT
CDR2 (VL):

(SEQ ID NO: 21)

SAS
CDR3 (VL):

(SEQ ID NO: 22)

QQYDSYPFT

Results—mAb#34 Sequences:

Heavy chain variable region (VH): amino acid sequence (SEQ ID NO:23)

EVQLQQSGPDLVKPGASVKISCKASGYSFTSYYMHWVKQSHGKSLEWIG
RVNPNSGYTTYNQKFKGKAILTVDKSSSTAYMELRSLTSEDSAVYYCAR
GDYLPSMDYWGHGTSVTVSS

Heavy chain variable region (VH): nucleotide sequence (SEQ ID NO:24)

GAGGTCCAGCTGCAGCAGTCTGGACCTGACCTGGTGAAGCCTGGGGCTT
CAGTGAAGATATCCTGCAAGGCTTCTGGTTACTCATTCACTAGCTACTA
CATGCACTGGGTGAAGCAGAGCCATGGAAAGAGCCTTGAGTGGATTGGC
CGTGTTAATCCTAACAGTGGTTATACTACCTACAACCAGAAGTTCAAGG
GCAAGGCCATATTAACTGTAGACAAGTCATCCAGCACAGCCTACATGGA
GCTCCGCAGCCTGACATCTGAGGACTCTGCGGTCTATTACTGTGCAAGG
GGGGATTACCTACCCTCTATGGACTACTGGGGTCATGGAACCTCAGTCA
CCGTCTCCTCA

Heavy chain variable region (VH)—CDR analysis:

----CDR1---> <--CDR2--> <---CDR3----
GYSFTSYY.... VNPNSGYT.. ARGDYLPSMDY
CDR1 (VH):

(SEQ ID NO: 25)

GYSFTSYY
CDR2 (VH):

(SEQ ID NO: 26)

VNPNSGYT
CDR3 (VH):

(SEQ ID NO: 27)

ARGDYLPSMDY

Light chain variable region (VL): amino acid sequence (SEQ ID NO:28)

DFVMTQSQKFMSTSVGDRVSITCKASQNVGTNVAWYQQKPGQSPKGLIY
SASYRYSGVPDRFTGSGSGTDFTLTISNVQSEDLAEYFCQQFNSYPFTF
GSGTKLEIK

Light chain variable region (VL): amino acid sequence (SEQ ID NO:29)

GACTTTGTGATGACCCAGTCTCAAAAATTCATGTCCACATCAGTAGGAG
ACAGGGTCAGCATCACCTGCAAGGCCAGTCAGAACGTGGGTACTAATGT
AGCCTGGTATCAACAGAAACCAGGGCAATCTCCTAAAGGATTGATTTAC
TCGGCATCCTACCGGTACAGTGGAGTCCCTGATCGCTTCACAGGTAGTG
GATCTGGGACAGATTTCACTCTCACCATCAGTAATGTGCAGTCTGAAGA
CTTGGCAGAGTATTTCTGTCAGCAATTTAACAGCTATCCATTCACGTTC
GGCTCGGGGACAAAGTTGGAAATAAAAC

Light chain variable region (VL)—CDR analysis:

----CDR1---> <--CDR2--> <---CDR3----
QNVGTN...... SAS....... QQFNSYPFT
CDR1 (VL):

(SEQ ID NO: 30)

QNVGTN
CDR2 (VL):

(SEQ ID NO: 31)

SAS
CDR3 (VL):

(SEQ ID NO: 32)

QQFNSYPFT

Comparison of CDR sequences between mAbs #2, 6 and 34

CDR1 (VH):

(SEQ ID NO: 5)

GYTFTSYW
CDR1 (VH):

(SEQ ID NO: 5)

GYSFTAYY
CDR1 (VH):

(SEQ ID NO: 25)

GYSFTSYY
CDR2 (VH):

(SEQ ID NO: 6)

IDPSDSES
CDR2 (VH):

(SEQ ID NO: 16)

VNPNNGGS
CDR2 (VH):

(SEQ ID NO: 26)

VNPNSGYT
CDR3 (VH):

(SEQ ID NO: 7)

ARKDYYAYGGAMDY
CDR3 (VH):

(SEQ ID NO: 17)

ARGDYLPSLGY
CDR3 (VH):

(SEQ ID NO: 27)

ARGDYLPSMDY
CDR1 (VL):

(SEQ ID NO: 10)

SSARY
CDR1 (VL):

(SEQ ID NO: 20)

QNVDTT
CDR1 (VL):

(SEQ ID NO: 30)

QNVGTN
CDR2 (VL):

(SEQ ID NO: 11)

STS
CDR2 (VL):

(SEQ ID NO: 21)

SAS
CDR2 (VL):

(SEQ ID NO: 31)

SAS
CDR3 (VL):

(SEQ ID NO: 12)

HQWSGYPT
CDR3 (VL):

(SEQ ID NO: 22)

QQYDSYPFT
CDR3 (VL):

(SEQ ID NO: 32)

QQFNSYPFT

Underlined sequences/amino acids indicate identical amino acids between CDRs.

As it appears, 9 of 11 amino acids (81.8%) of CDR3 (VH) of mAb#6 and #34 are identical. Thus, a VH CDR3 sequence having the sequence ARGDYLPSXXY (SEQ ID NO:33), wherein XX denotes any amino acid, may be expected to have the same function as SEQ ID NO: 17 and 27. In one embodiment XX is selected from LG or MD.

7 of 8 amino acids (87.5%) of CDR1 (VH) of mAb#6 and #34 are identical. Thus, a VH CDR1 sequence having the sequence GYSFTXYY (SEQ ID NO:34), wherein X denotes any amino acid, may be expected to have the same function as SEQ ID NO: 15 and 25. In one embodiment, X is A or S.

7 of 9 amino acids (77.8%) of CDR3 (VL) of mAb#6 and #34 are identical. Thus, a LH CDR3 sequence having the sequence QQXXSYPFT (SEQ ID NO:35), wherein XX denotes any amino acid, may be expected to have the same function as SEQ ID NO: 22 and 32. In one embodiment XX is selected from YD or FN.

1-16. (canceled)

17. A method of treating a subject suffering from an autoimmune disease characterized by extracellular citrullination comprising the administration of a suitable amount of an anti-PAD2 antibody to said subject.

18. The method according to claim 17, wherein said autoimmune disease is selected from the group consisting of rheumatoid arthritis, multiple sclerosis, Sjögren's syndrome and psoriasis.

19. The method according to claim 17, wherein said autoimmune disease is rheumatoid arthritis.

20. The method according to claim 17, wherein said treatment is prophylactic, ameliorative or curative.

21. The method according to claim 19, wherein said treatment is a prophylactic treatment initiated upon detection of ACPAs in said subject.

22. The method according to claim 17, wherein said anti-PAD2 antibody is co-administered with another drug suitable for treating said autoimmune disease.

23. The method according to claim 17, wherein the subject is human.

24. (canceled)

25. An anti-PAD2 antibody according to claim 2, comprising
i) a Heavy chain variable region (VH) selected from the group consisting of SEQ ID NO:3, SEQ ID NO:13 and SEQ ID NO:23, or a variant thereof having at least 75% sequence identity thereto;
and/or comprises
ii) a Light chain variable region (VL) selected from the group consisting of SEQ ID NO:8, SEQ ID NO:18 and SEQ ID NO:28, or a variant thereof having at least 75% sequence identity thereto.

26. The anti-PAD2 antibody according to claim 25, wherein said antibody is selected from an antibody
i) comprising a Heavy chain variable region (VH) of sequence SEQ ID NO:3, or a variant thereof having at least 75% sequence identity thereto; and/or comprising a Light chain variable region (VL) of sequence SEQ ID NO:8, or a variant thereof having at least 75% sequence identity thereto;
ii) comprising a Heavy chain variable region (VH) of sequence SEQ ID NO:13, or a variant thereof having at least 75% sequence identity thereto; and/or comprising a Light chain variable region (VL) of sequence SEQ ID NO:18, or a variant thereof having at least 75% sequence identity thereto; and
iii) comprising a Heavy chain variable region (VH) of sequence SEQ ID NO:23, or a variant thereof having at least 75% sequence identity thereto; and/or comprising a Light chain variable region (VL) of sequence SEQ ID NO:28, or a variant thereof having at least 75% sequence identity thereto.

27. The anti-PAD2 antibody according to claim 25, wherein said variant has at least 80%, 85%, 90%, 95%, 96%, 97%, 98% or 99% sequence identity to a Heavy chain variable region (VH) of sequence SEQ ID NO:3, SEQ ID NO:13 or SEQ ID NO:23; or a Light chain variable region (VL) of sequence SEQ ID NO:8, SEQ ID NO:18 or SEQ ID NO:28.

28. The anti-PAD2 antibody according to claim 25, wherein said antibody comprises
i) a Heavy chain variable region (VH) comprising one, two or three binding domains selected from
1) a first binding domain comprising a VH CDR1 selected from the group consisting of SEQ ID NO:5, SEQ ID NO:15 and SEQ ID NO:25, or a sequence having at least 75% sequence identity thereto;
2) a second binding domain comprising a VH CDR2 selected from the group consisting of SEQ ID NO:6, SEQ ID NO:16 and SEQ ID NO:26, or a sequence having at least 75% sequence identity thereto; and
3) a third binding domain comprising a VH CDR3 selected from the group consisting of SEQ ID NO:7, SEQ ID NO:17 and SEQ ID NO:27, or a sequence having at least 75% sequence identity thereto;
and/or comprises
ii) a Light chain variable region (VL) comprising one, two or three binding domains selected from
1) a first binding domain comprising a VL CDR1 selected from the group consisting of SEQ ID NO:10, SEQ ID NO:20 and SEQ ID NO:30, or a sequence having at least 75% sequence identity thereto;
2) a second binding domain comprising a VL CDR2 selected from the group consisting of SEQ ID NO:11, SEQ ID NO:21 and SEQ ID NO:31, or a sequence having at least 75% sequence identity thereto; and
3) a third binding domain comprising a VL CDR3 selected from the group consisting of SEQ ID NO:12, SEQ ID NO:22 and SEQ ID NO:32, or a sequence having at least 75% sequence identity thereto.

29. The anti-PAD2 antibody according to claim 28, wherein said variant has at least 80%, 85%, 90%, 95%, 96%, 97%, 98% or 99% sequence identity to a VH CDR1 selected from the group consisting of SEQ ID NO:5, SEQ ID NO:15 and SEQ ID NO:25; a VH CDR2 selected from the group consisting of SEQ ID NO:6, SEQ ID NO:16 and SEQ ID NO:26; a VH CDR3 selected from the group consisting of SEQ ID NO:7, SEQ ID NO:17 and SEQ ID NO:27; a VL CDR1 selected from the group consisting of SEQ ID NO:10, SEQ ID NO:20 and SEQ ID NO:30; a VL CDR2 selected from the group consisting of SEQ ID NO:11, SEQ ID NO:21 and SEQ ID NO:31; or a VL CDR3 selected from the group consisting of SEQ ID NO:12, SEQ ID NO:22 and SEQ ID NO:32.

30. The anti-PAD2 antibody according to claim 25, wherein said antibody comprises a Heavy chain variable region (VH) comprising a VH CDR3 selected from the group consisting of SEQ ID NO:7, SEQ ID NO:17, SEQ ID NO:27 and SEQ ID NO:33, wherein said antibody inhibits PAD2-catalyzed citrullination of a substrate such as fibrinogen.

31. The anti-PAD2 antibody according claim 25, wherein said antibody comprises
i) a Heavy chain variable region (VH) comprising one, two or three binding domains selected from 1) a first binding domain comprising a VH CDR1 selected from the group consisting of SEQ ID NO:5, SEQ ID NO:15 and SEQ ID NO:25; 2) a second binding domain comprising a VH CDR2 selected from the group consisting of SEQ ID NO:6, SEQ ID NO:16 and SEQ ID NO:26; and 3) a third binding domain comprising a VH CDR3 selected from the group consisting of SEQ ID NO:7, SEQ ID NO:17 and SEQ ID NO:27;
and comprises
ii) a Light chain variable region (VL) comprising one, two or three binding domains selected from 1) a first binding domain comprising a VL CDR1 selected from the group consisting of SEQ ID NO:10, SEQ ID NO:20 and SEQ ID NO:30; 2) a second binding domain comprising a VL CDR2 selected from the group consisting of SEQ ID NO:11, SEQ ID NO:21 and SEQ ID NO:31; and 3) a third binding domain comprising a VL CDR3 selected from the group consisting of SEQ ID NO:12, SEQ ID NO:22 and SEQ ID NO:32.

32. An anti-PAD2 antibody that recognizes and specifically binds to the same epitope as an anti-PAD2 antibody selected from the group consisting of an antibody comprising a VH domain identified as SEQ ID NO:3 and a VL domain identified as SEQ ID NO:8; an antibody comprising a VH domain identified as SEQ ID NO:13 and a VL domain identified as SEQ ID NO:18; and an antibody comprising a VH domain identified as SEQ ID NO:23 and a VL domain identified as SEQ ID NO:28.

33. (canceled)

34. The anti-PAD2 antibody according to claim 25, wherein said antibody inhibits enzyme activity of PAD2 and/or increases the clearance of PAD2.

35. The anti-PAD2 antibody according to claim 25, wherein said antibody inhibits citrullination of fibrinogen with human recombinant PAD2 (hrPAD2) as catalyst.

36. The anti-PAD2 antibody according to claim 25, wherein the antibody is a fully non-human antibody, a chimeric antibody, a humanized antibody or a fully human antibody.

37-38. (canceled)

39. The method according to claim 17, wherein the anti-PAD2 antibody is as defined in claim 25.

40. A pharmaceutical composition comprising an anti-PAD2 antibody according to claim 25 and at least one pharmaceutically acceptable diluent, carrier or excipient.


SEQUENCE LISTING (35 sequences)

SEQ ID NO: 1 - protein, 665 aa, Homo sapiens
MLRERTVRLQYGSRVEAVYVLGTYLWTDVYSAAPAGAQTFSLKHSEHVWVEVVRDGEAEEVATNGKQRWLLSPSTTLRVTMSQASTEASSDKVTVNYYDEEGSIPIDQAGLFLTAIEISLDVDADRDGVVEKNNPKKASWTWGPEGQGAILLVNCDRETPWLPKEDCRDEKVYSKEDLKDMSQMILRTKGPDRLPAGYEIVLYISMSDSDKVGVFYVENPFFGQRYIHILGRRKLYHVVKYTGGSAELLFFVEGLCFPDEGFSGLVSIHVSLLEYMAQDIPLTPIFTDTVIFRIAPWIMTPNILPPVSVFVCCMKDNYLFLKEVKNLVEKTNCELKVCFQYLNRGDRWIQDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWWHMVP

SEQ ID NO: 2 - protein, 665 aa, Oryctolagus cuniculus
MPRERTVRLQYGSRVEAVYVLGTPLWIDVYSAAPAEARTFSLKHSERVRVEVVRDGEAEEVATNGKQRWLLSPSTTLRLTMGQASTEASSDKVTVNYYDEEGNAPIDQAGLFLTAIEISLDVDADRDGVVEKNNPQKASWTWGPEGQGAILLVNCDRDTPWLPKEDCSDEKVYSKEDLKDMSQMILRTKGPDRLPAGYEMVLYISVSDSDKVGVFYVENPFFGQRYIHILGRRKLYHVVKYTGGSAELLFFVEGLRFPDEGFSGLVSIHVSLLEYMAEEVPLTPIFTDTVTFRIAPWIMTPNILPPVSVFVCCMKDNYLFLKEVKNLVEKTNCELKVCFQYMNRGDRWIQDEIEFGYIEAPHKGFPVVLDSPRDGNLKDFPIKQLLGPDFGYVTREPVFETVTSLDSFGNLEVSPPVTVNGKTYPLGRILIGSSFPLSGGRRMTKVVRDFLQAQQVQAPVELYSDWLTVGHVDEFMTFVPIPGTTQFRLLMASTAACYKLFREKQKEGHGEAILFKGLGGMSSKRITINKVLSNESLAQENQYFQRCLDWNRDILKKELGLTEQDIIDLPALFKMDEGRQARAFFPNMVNMIVLNKDLGIPKPFGPQVEEECCLETHVRGLLEPLGLACTFIDDISAYHKFLGEVHCGTNVHRKPFTFKWWHMVP

SEQ ID NO: 3 - protein, 121 aa, Mus musculus
QVQLQQPGAELVRPGASVKLSCKASGYTFTSYWMNWVKQRPGQGLEWIGMIDPSDSESYYNQMFKDKATLTVDKSSSTAYMQLSRLTSEDSAVYYCARKDYYAYGGAMDYWGQGTSVTVSS

SEQ ID NO: 4 - DNA, 363 bp, Mus musculus
caggtccaactgcagcagcctggggctgaactggtgaggcctggggcttcagtgaagctgtcctgcaaggcttctggctacaccttcaccagctactggatgaactgggtgaagcagaggcctggacaaggccttgaatggattggtatgattgatccttcagacagtgaaagttattacaatcaaatgttcaaggacaaggccacattgactgtagacaaatcctccagcacagcctacatgcagctcagcaggttgacatctgaggactctgcggtctattactgtgcaagaaaggattactacgcctacgggggagctatggactactggggtcaaggaacctcagtcaccgtctcctca

SEQ ID NO: 5 - protein, 8 aa, Mus musculus
GYTFTSYW

SEQ ID NO: 6 - protein, 8 aa, Mus musculus
IDPSDSES

SEQ ID NO: 7 - protein, 14 aa, Mus musculus
ARKDYYAYGGAMDY

SEQ ID NO: 8 - protein, 106 aa, Mus musculus
QIVLTQSPAILSASLGEEITLTCSANSSARYMHWYQQKSGTSPKLLIYSTSNLASGVPSRFSGSGSGTFYSLTLSSVEAEDAADYYCHQWSGYPTFGGGTKLEIKR

SEQ ID NO: 9 - DNA, 317 bp, Mus musculus
cagattgttctcacccagtctccagcaatcctgtctgcatctctaggggaggagatcaccctaacctgcagtgccaactcgagtgcacgttacatgcactggtaccagcagaagtcaggcacttctcccaaactcttgatttatagcacatccaacctggcttctggagtcccttctcgcttcagtggcagtgggtctgggaccttttattctctcacactcagcagtgtggaggctgaagatgctgccgattattactgccatcagtggagtggttatcccacgttcggaggggggaccaagctggaaataaaacg

SEQ ID NO: 10 - protein, 5 aa, Mus musculus
SSARY

SEQ ID NO: 11 - protein, 3 aa, Mus musculus
STS

SEQ ID NO: 12 - protein, 8 aa, Mus musculus
HQWSGYPT

SEQ ID NO: 13 - protein, 118 aa, Mus musculus
EVQLQQSGPDLVKPGASVKISCKASGYSFTAYYMHWVKQSHGESLEWIGRVNPNNGGSSYNQKFKGKAILTVHKSSNTAYMELRSLTSEDSAVYFCARGDYLPSLGYWGQGTSVTVSS

SEQ ID NO: 14 - DNA, 354 bp, Mus musculus
gaggtccagctgcagcagtctggacctgacctggtgaagcctggggcttcagtgaagatatcctgcaaggcttctggttactcattcactgcctactacatgcactgggtgaagcagagccatggagagagccttgagtggattggacgtgttaatcctaacaatggtggtagtagctacaaccagaagttcaagggcaaggccatattaactgtacacaagtcatccaacacagcctacatggagctccgcagcctgacatctgaggactctgcggtctatttctgtgcaaggggggattaccttccctctttgggctactggggtcaaggaacctcagtcaccgtctcctca

SEQ ID NO: 15 - protein, 8 aa, Mus musculus
GYSFTAYY

SEQ ID NO: 16 - protein, 8 aa, Mus musculus
VNPNNGGS

SEQ ID NO: 17 - protein, 11 aa, Mus musculus
ARGDYLPSLGY

SEQ ID NO: 18 - protein, 107 aa, Mus musculus
DIVMTQSQKFMSTSVGDRVSVTCKASQNVDTTVAWYQQKPGQSPKALIYSASYRYSGVPDRFTGSGSGTDFTLTVTNVQSEDLAEYFCQQYDSYPFTFGSGTKLEIK

SEQ ID NO: 19 - DNA, 322 bp, Mus musculus
gacattgtgatgacccagtctcaaaaattcatgtccacatcagtaggagacagggtcagcgtcacctgcaaggccagtcagaatgtggatactactgtagcctggtatcaacagaaaccaggacaatctcctaaagcactgatttactcggcatcctaccggtacagtggagtccctgatcgcttcacaggcagtggatctgggacagatttcactctcaccgtcaccaatgtgcagtctgaagacttggcagagtatttctgtcagcaatatgacagctatccattcacgttcggctcggggacaaagttggaaataaaac

SEQ ID NO: 20 - protein, 6 aa, Mus musculus
QNVDTT

SEQ ID NO: 21 - protein, 3 aa, Mus musculus
SAS

SEQ ID NO: 22 - protein, 9 aa, Mus musculus
QQYDSYPFT

SEQ ID NO: 23 - protein, 118 aa, Mus musculus
EVQLQQSGPDLVKPGASVKISCKASGYSFTSYYMHWVKQSHGKSLEWIGRVNPNSGYTTYNQKFKGKAILTVDKSSSTAYMELRSLTSEDSAVYYCARGDYLPSMDYWGHGTSVTVSS

SEQ ID NO: 24 - DNA, 354 bp, Mus musculus
gaggtccagctgcagcagtctggacctgacctggtgaagcctggggcttcagtgaagatatcctgcaaggcttctggttactcattcactagctactacatgcactgggtgaagcagagccatggaaagagccttgagtggattggccgtgttaatcctaacagtggttatactacctacaaccagaagttcaagggcaaggccatattaactgtagacaagtcatccagcacagcctacatggagctccgcagcctgacatctgaggactctgcggtctattactgtgcaaggggggattacctaccctctatggactactggggtcatggaacctcagtcaccgtctcctca

SEQ ID NO: 25 - protein, 8 aa, Mus musculus
GYSFTSYY

SEQ ID NO: 26 - protein, 8 aa, Mus musculus
VNPNSGYT

SEQ ID NO: 27 - protein, 11 aa, Mus musculus
ARGDYLPSMDY

SEQ ID NO: 28 - protein, 107 aa, Mus musculus
DFVMTQSQKFMSTSVGDRVSITCKASQNVGTNVAWYQQKPGQSPKGLIYSASYRYSGVPDRFTGSGSGTDFTLTISNVQSEDLAEYFCQQFNSYPFTFGSGTKLEIK

SEQ ID NO: 29 - DNA, 322 bp, Mus musculus
gactttgtgatgacccagtctcaaaaattcatgtccacatcagtaggagacagggtcagcatcacctgcaaggccagtcagaacgtgggtactaatgtagcctggtatcaacagaaaccagggcaatctcctaaaggattgatttactcggcatcctaccggtacagtggagtccctgatcgcttcacaggtagtggatctgggacagatttcactctcaccatcagtaatgtgcagtctgaagacttggcagagtatttctgtcagcaatttaacagctatccattcacgttcggctcggggacaaagttggaaataaaac

SEQ ID NO: 30 - protein, 6 aa, Mus musculus
QNVGTN

SEQ ID NO: 31 - protein, 3 aa, Mus musculus
SAS

SEQ ID NO: 32 - protein, 9 aa, Mus musculus
QQFNSYPFT

SEQ ID NO: 33 - protein, 11 aa, Mus musculus
  VARIANT (9)..(10): XX denotes any amino acid or two consecutive amino acids selected from LG and MD
ARGDYLPSXXY

SEQ ID NO: 34 - protein, 8 aa, Mus musculus
  VARIANT (6)..(6): X denotes any amino acid or an amino acid selected from A and S
GYSFTXYY

SEQ ID NO: 35 - protein, 9 aa, Mus musculus
  VARIANT (3)..(4): XX denotes any amino acid or two consecutive amino acids selected from YD and FN
QQXXSYPFT